(19) United States
(12) Patent Application Publication
(10) Pub. No.: US 2012/0183949 A1
(43) Pub. Date: Jul. 19, 2012

(54) Method, device, or system using lung sensor for detecting a physiological condition in a vertebrate subject

(75) Inventors: Roderick A. Hyde, Redmond, WA (US); Muriel Y. Ishikawa, Livermore, CA (US); Jordin T. Kare, Seattle, WA (US); Erez Lieberman, Cambridge, MA (US); Eric C. Leuthardt, St. Louis, MO (US); Lowell L. Wood, JR., Bellevue, WA (US); Victoria Y.H. Wood, Livermore, CA (US)

(73) Assignee: Searete LLC, a limited liability corporation of the State of Delaware

(21) Appl. No.: 12/930,968

(22) Filed: Jan. 19, 2011

(51) Int. Cl. G01N 33/53; C12Q 1/02; G01N 1/22; C12M 1/34; C12Q 1/70; G01N 27/72; B82Y 15/00

(52) U.S. Cl. 435/5; 436/501; 435/34; 422/84; 435/287.1; 977/773; 977/838; 977/904; 977/774

(57) ABSTRACT

Devices, systems, and methods are disclosed herein for detecting one or more physiological conditions in lungs of a vertebrate subject. A method is described for administering at least one microparticle to lungs of a vertebrate subject, wherein the at least one microparticle includes one or more markers; wherein the one or more markers is configured to be released in response to one or more physiological conditions in the vertebrate subject; and detecting the one or more markers in a lung exhalant of the vertebrate subject.

### SUMMARY

Devices, systems, and methods are disclosed herein for detecting one or more physiological conditions in the lungs of a vertebrate subject. A method is described herein that includes administering at least one microparticle to lungs of a vertebrate subject, wherein the at least one microparticle includes one or more markers; wherein the one or more markers is configured to be released in response to one or more physiological conditions in the vertebrate subject; and detecting the one or more markers in a lung exhalant of the vertebrate subject.

The method can further include determining the existence or absence of the one or more physiological conditions in the vertebrate subject. The one or more physiological conditions can include at least one of lung tissue pH, lung chemical environment, lung tissue protein expression, lung bacterial occupation, or lung viral occupation. The one or more physiological conditions can include one or more aspects of blood condition or blood chemistry. The one or more markers can include at least one of a gas, vapor-phase chemical, gas-entrained aerosol, gas-entrained chemical, gas-entrained nanoparticle, or gas-entrained magnetic nanoparticle. The gas-entrained nanoparticle or the gas-entrained magnetic nanoparticle can include one or more quantum dots. The gas-entrained nanoparticle or the gas-entrained magnetic nanoparticle can include one or more of optically responsive components, dyes, fluorescent dyes, rare earth metals, or nonlinear optical materials. The gas-entrained nanoparticle or the gas-entrained magnetic nanoparticle can include one or more of plasmon responsive components, plasmon responsive metallic structure, or plasmon responsive metallic coating. The one or more markers can be embedded within the at least one microparticle or within an internal compartment of the at least one microparticle. The method can further include measuring the one or more markers in the lung exhalant based on at least one of odor, spectrometry, fluorescence spectrometry, chemical interaction, plasmon interaction, mass spectrometry, magnetism, or radioactivity. The at least one microparticle can include one or more of liposomes, microspheres, hydrogels, or porous nanoparticle-aggregate particles (PNAP). The at least one microparticle can be configured to release the one or more markers in response to a lung tissue environment or lung cell interaction. The at least one microparticle can be configured to release the one or more markers based on at least one of lung tissue pH, lung chemical environment, cell surface receptor binding, endocytosis, interaction with vertebrate cells, interaction with bacterial cells, or interaction with viruses or viral-infected cells, in the lung tissue of the vertebrate subject. The at least one microparticle can be configured to release the one or more markers based on one or more of blood condition or blood chemistry.

The method can further include providing one or more sensors configured to detect the one or more markers in the lung exhalant. The one or more sensors can be in communication with one or more controllers. The one or more controllers can be configured to regulate a time period for detecting the one or more markers in the lung exhalant from the vertebrate subject. The time period for detecting can be substantially immediately upon sensing the exhalant or delayed upon sensing the exhalant. The time period for detecting can be a predetermined time after the administration of the at least one microparticle.

The method can further include providing at least one chemical trigger or physical trigger configured to expose the one or more markers within the at least one microparticle. The at least one chemical trigger or physical trigger can be configured to expose the one or more markers to the physiological conditions surrounding the at least one microparticle. The at least one chemical trigger or physical trigger can be a coating at least partially surrounding the at least one microparticle. The at least one chemical trigger or physical trigger can be configured to be responsive to at least one of lung tissue pH, lung chemical environment, cell surface receptor binding, endocytosis, cellular interaction with vertebrate cells, cellular interaction with bacterial cells, cellular interaction with viruses or viral-infected cells, or transdermal energy deposition, in the lung tissue of the vertebrate subject. The method can further include providing one or more sensors that detect the one or more markers in the lung exhalant, wherein the one or more sensors is in communication with one or more controllers. The one or more controllers can be configured to activate the at least one chemical trigger or physical trigger to expose the one or more markers within the at least one microparticle for detection by the one or more sensors. The one or more controllers can be configured to activate the at least one chemical trigger or physical trigger by transdermal energy deposition. The one or more controllers can be configured to activate the at least one chemical trigger or physical trigger in a targeted lung region of the vertebrate subject. The method can further include providing the at least one microparticle having at least one hydraulic diameter configured to interact at one or more depths in the lung tissue of the vertebrate subject. The at least one microparticle can include at least one magnetic microparticle. The method can further include providing one or more magnetic elements configured to control motion of the at least one magnetic microparticle.

A device is described herein that includes a device including an applicator configured to deliver at least one microparticle to lungs of a vertebrate subject, wherein the at least one microparticle includes one or more markers; and wherein the one or more markers is configured to be released in response to one or more physiological conditions in the vertebrate subject; and one or more sensors configured to detect the one or more markers in a lung exhalant of the vertebrate subject. The detection of the one or more markers in the lung exhalant can be indicative of the existence or absence of the one or more physiological conditions in the vertebrate subject. The applicator can include one or more of an inhaler, atomizer, nebulizer, aerosolizer, mister, dry powder inhaler, metered dose inhaler, metered dose sprayer, metered dose mister, or metered dose atomizer. The device including the applicator and the one or more sensors can be in one unit. The device including the applicator and the one or more sensors can be in two or more separate units. The one or more physiological conditions can include at least one of lung tissue pH, lung chemical environment, cell surface receptor binding, endocytosis, cellular interaction with vertebrate cells, or cellular interaction with bacterial cells. The one or more physiological conditions can include one or more aspects of blood condition or blood chemistry. The one or more markers can include at least one of a gas, vapor-phase chemical, gas-entrained aerosol, gas-entrained chemical, or gas-entrained nanoparticle, or gas-entrained magnetic nanoparticle. The gas-entrained nanoparticle or the gas-entrained magnetic nanoparticle can include one or more quantum dots. The gas-entrained nanoparticle or the gas-entrained magnetic nanoparticle can include one or more of optically responsive components, dyes, fluorescent dyes, rare earth metals, or nonlinear optical materials. The gas-entrained nanoparticle or the gas-entrained magnetic nanoparticle can include one or more of plasmon responsive components, plasmon responsive metallic structure, or plasmon responsive metallic coating. The one or more markers can be embedded within the at least one microparticle or within an internal compartment of the at least one microparticle. The one or more markers in the lung exhalant can be configured to be measured based on at least one of odor, spectrometry, fluorescence spectrometry, chemical interaction, plasmon interaction, mass spectrometry, or radioactivity. The at least one microparticle can include one or more of liposomes, microspheres, hydrogels, or porous nanoparticle-aggregate particles (PNAP). The at least one microparticle can be configured to release the one or more markers in response to a lung tissue environment or lung cell interaction. The at least one microparticle can be configured to release the one or more markers based on at least one of lung tissue pH, lung chemical environment, cell surface receptor binding, endocytosis, interaction with vertebrate cells, interaction with bacterial cells, or interaction with viruses or viral-infected cells, in the lung tissue of the vertebrate subject. The at least one microparticle can be configured to release the one or more markers based on one or more of blood condition or blood chemistry. The one or more sensors can be in communication with one or more controllers. The one or more controllers can be configured to regulate a time period for detecting the one or more markers in the lung exhalant from the vertebrate subject. The time period for detecting can be substantially immediately upon sensing the exhalant or delayed upon sensing the exhalant. The time period for detecting can be a predetermined time after the administration of the at least one microparticle.

The device can further include at least one chemical trigger or physical trigger configured to expose the one or more markers within the at least one microparticle. The at least one chemical trigger or physical trigger can be configured to expose the one or more markers to the physiological conditions surrounding the at least one microparticle. The at least one chemical trigger or physical trigger can be a coating at least partially surrounding the at least one microparticle. The at least one chemical trigger or physical trigger can be configured to be responsive to at least one of lung tissue pH, lung chemical environment, cell surface receptor binding, endocytosis, cellular interaction with vertebrate cells, or cellular interaction with bacterial cells, cellular interaction with viruses or viral-infected cells, or transdermal energy deposition, in the lung tissue of the vertebrate subject. The device can further include one or more controllers configured to activate the at least one chemical trigger or physical trigger to expose the one or more markers within the at least one microparticle for detection by the one or more sensors. The one or more controllers can be configured to activate the at least one chemical trigger or physical trigger by transdermal energy deposition. The one or more controllers can be configured to activate the at least one chemical trigger or physical trigger in a targeted lung region of the vertebrate subject. The at least one microparticle having at least one hydraulic diameter can be configured to interact at one or more depths in the lung tissue of the vertebrate subject. The at least one microparticle can include at least one magnetic microparticle. The device can further include one or more magnetic elements configured to control motion of the at least one magnetic microparticle. The magnetic element can be a separate unit from the applicator and the one or more sensors. The device can further include one or more controllers configured to regulate a time period for detection of the one or more markers in the lung exhalant of the vertebrate subject. The time period for detection can be immediately upon sensing the exhalant or delayed upon sensing the exhalant.

A system is described herein that includes at least one microparticle in a pharmaceutical composition; a device including an applicator configured to deliver the at least one microparticle in the pharmaceutical composition to lungs of a vertebrate subject, wherein the at least one microparticle includes one or more markers; and wherein the one or more markers is configured to be released in response to one or more physiological conditions in the vertebrate subject; and one or more sensors configured to detect the one or more markers in a lung exhalant of the vertebrate subject.

The detection of the one or more markers in the lung exhalant can be indicative of the existence or absence of the one or more physiological conditions in the vertebrate subject. The applicator can include one or more of an inhaler, atomizer, nebulizer, aerosolizer, mister, dry powder inhaler, metered dose inhaler, metered dose sprayer, metered dose mister, or metered dose atomizer. The device including the applicator and the one or more sensors can be in one unit. The device including the applicator and the one or more sensors can be in two or more separate units. The one or more physiological conditions can include at least one of lung tissue pH, lung chemical environment, cell surface receptor binding, endocytosis, cellular interaction with vertebrate cells, or cellular interaction with bacterial cells. The one or more physiological conditions can include one or more aspects of blood condition or blood chemistry. The one or more markers can include at least one of a gas, vapor-phase chemical, gas-entrained aerosol, gas-entrained chemical, or gas-entrained nanoparticle, or gas-entrained magnetic nanoparticle. The gas-entrained nanoparticle or the gas-entrained magnetic nanoparticle can include one or more quantum dots. The gas-entrained nanoparticle or the gas-entrained magnetic nanoparticle can include one or more of optically responsive components, dyes, fluorescent dyes, rare earth metals, or nonlinear optical materials. The gas-entrained nanoparticle or the gas-entrained magnetic nanoparticle can include one or more of plasmon responsive components, plasmon responsive metallic structure, or plasmon responsive metallic coating. The one or more markers can be embedded within the at least one microparticle or within an internal compartment of the at least one microparticle. The one or more markers in the lung exhalant can be configured to be measured based on at least one of odor, spectrometry, fluorescence spectrometry, chemical interaction, plasmon interaction, mass spectrometry, or radioactivity. The at least one microparticle can include one or more of liposomes, microspheres, hydrogels, or porous nanoparticle-aggregate particles (PNAP). The at least one microparticle can be configured to release the one or more markers in response to a lung tissue environment or lung cell interaction. The at least one microparticle can be configured to release the one or more markers based on at least one of lung tissue pH, lung chemical environment, cell surface receptor binding, endocytosis, interaction with vertebrate cells, interaction with bacterial cells, or interaction with viruses or viral-infected cells, in the lung tissue of the vertebrate subject. The at least one microparticle can be configured to release the one or more markers based on one or more of blood condition or blood chemistry. The one or more sensors can be in communication with one or more controllers. The one or more controllers can be configured to regulate a time period for detecting the one or more markers in the lung exhalant from the vertebrate subject. The time period for detecting can be substantially immediately upon sensing the exhalant or delayed upon sensing the exhalant. The time period for detecting can be a predetermined time after the administration of the at least one microparticle.

The system can further include at least one chemical trigger or physical trigger configured to expose the one or more markers within the at least one microparticle. The at least one chemical trigger or physical trigger can be configured to expose the one or more markers to the physiological conditions surrounding the at least one microparticle. The at least one chemical trigger or physical trigger can be a coating at least partially surrounding the at least one microparticle. The at least one chemical trigger or physical trigger can be configured to be responsive to at least one of lung tissue pH, lung chemical environment, cell surface receptor binding, endocytosis, cellular interaction with vertebrate cells, or cellular interaction with bacterial cells, cellular interaction with viruses or viral-infected cells, or transdermal energy deposition, in the lung tissue of the vertebrate subject. The system can further include one or more controllers configured to activate the at least one chemical trigger or physical trigger to expose the one or more markers within the at least one microparticle for detection by the one or more sensors. The one or more controllers can be configured to activate the at least one chemical trigger or physical trigger by transdermal energy deposition. The one or more controllers can be configured to activate the at least one chemical trigger or physical trigger in a targeted lung region of the vertebrate subject. The at least one microparticle having at least one hydraulic diameter can be configured to interact at one or more depths in the lung tissue of the vertebrate subject. The at least one microparticle can include at least one magnetic microparticle. The system can further include one or more magnetic elements configured to control motion of the at least one magnetic microparticle. The magnetic element can be a separate unit from the applicator and the one or more sensors. The device can further include one or more controllers configured to regulate a time period for detection of the one or more markers in the lung exhalant of the vertebrate subject. The time period for detection can be immediately upon sensing the exhalant or delayed upon sensing the exhalant.

A method is described herein that includes means for administering at least one microparticle to lungs of a vertebrate subject, wherein the at least one microparticle includes one or more markers; wherein the one or more markers is configured to be released in response to one or more physiological conditions in the vertebrate subject; and means for detecting the one or more markers in a lung exhalant of the vertebrate subject. The method can further include means for determining the existence or absence of the one or more physiological conditions in the vertebrate subject. The one or more physiological conditions can include one or more of lung tissue pH, lung chemical environment, lung tissue protein expression, lung bacterial occupation, or lung viral occupation. The one or more physiological conditions can include one or more aspects of blood condition or blood chemistry. The one or more markers can include at least one of a gas, vapor-phase chemical, gas-entrained aerosol, gas-entrained chemical, gas-entrained nanoparticle, or gas-entrained magnetic nanoparticle. The gas-entrained nanoparticle or the gas-entrained magnetic nanoparticle can include one or more quantum dots. The gas-entrained nanoparticle or the gas-entrained magnetic nanoparticle can include one or more of optically responsive components, dyes, fluorescent dyes, rare earth metals, or nonlinear optical materials. The gas-entrained nanoparticle or the gas-entrained magnetic nanoparticle can include one or more of plasmon responsive components, plasmon responsive metallic structure, or plasmon responsive metallic coating. The one or more markers can be embedded within the at least one microparticle or within an internal compartment of the at least one microparticle. The method can further include means for measuring the one or more markers in the lung exhalant based on at least one of odor, spectrometry, fluorescence spectrometry, chemical interaction, plasmon interaction, mass spectrometry, magnetism, or radioactivity. The at least one microparticle can include one or more of liposomes, microspheres, hydrogels, or porous nanoparticle-aggregate particles (PNAP).

The at least one microparticle can be configured to release the one or more markers in response to a lung tissue environment or lung cell interaction. The at least one microparticle can be configured to release the one or more markers based on at least one of lung tissue pH, lung chemical environment, cell surface receptor binding, endocytosis, interaction with vertebrate cells, interaction with bacterial cells, or interaction with viruses or viral-infected cells in the lung tissue of the vertebrate subject. The at least one microparticle can be configured to release the one or more markers based on one or more of blood condition or blood chemistry. The method can further include providing means for sensing configured to detect the one or more markers in the lung exhalant. The sensing means can be in communication with controlling means. The controlling means can be configured to regulate a time period for detecting the one or more markers in the lung exhalant from the vertebrate subject. The time period for detecting can be substantially immediately upon sensing the exhalant or delayed upon sensing the exhalant. The time period for detecting can be a predetermined time after the administration of the at least one microparticle.

The method can further include providing at least one chemical trigger means or physical trigger means configured to expose the one or more markers within the at least one microparticle. The at least one chemical trigger means or physical trigger means can be configured to expose the one or more markers to the physiological conditions surrounding the at least one microparticle. The chemical trigger means or the physical trigger means can be a coating at least partially surrounding the at least one microparticle. The at least one chemical trigger means or physical trigger means can be configured to be responsive to at least one of lung tissue pH, lung chemical environment, cell surface receptor binding, endocytosis, interaction with vertebrate cells, interaction with bacterial cells, interaction with viruses or viral-infected cells, or transdermal energy deposition in the lung tissue of the vertebrate subject. The method can further include providing sensor means that detect the one or more markers in the lung exhalant, wherein the sensor means is in communication with controller means. The controller means can be configured to activate the chemical trigger means or the physical trigger means to expose the one or more markers within the at least one microparticle for detection by the sensor means. The controller means can be configured to activate the chemical trigger means or the physical trigger means by transdermal energy deposition. The controller means can be configured to activate the chemical trigger means or the physical trigger means in a targeted lung region of the vertebrate subject. The at least one microparticle can include at least one microparticle having at least one hydraulic diameter configured to interact at one or more depths in the lung tissue of the vertebrate subject. The at least one microparticle can include at least one magnetic microparticle. The method can further include means for providing one or more magnetic elements configured to control motion of the at least one magnetic microparticle.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 depicts a diagrammatic view of an aspect of an embodiment of a device.

FIG. 2 depicts a diagrammatic view of an aspect of an embodiment of a device.

FIG. 3 depicts a diagrammatic view of an aspect of an embodiment of a method.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

This document uses formal outline headings for clarity of presentation. However, it is to be understood that the outline headings are for presentation purposes, and that different types of subject matter may be discussed throughout the application (e.g., method(s) may be described under composition heading(s) and/or kit headings, and/or descriptions of single topics may span two or more topic headings). Hence, the use of the formal outline headings is not intended to be in any way limiting.

Devices, systems, and methods are disclosed herein for detecting one or more physiological conditions in the lungs of a vertebrate subject. A method is described herein that includes administering at least one microparticle to lungs of a vertebrate subject, wherein the at least one microparticle includes one or more markers; wherein the one or more markers is configured to be released in response to one or more physiological conditions in the vertebrate subject, and detecting the one or more markers in a lung exhalant of the vertebrate subject. The method can further include determining the existence or absence of the one or more physiological conditions in the vertebrate subject. The one or more physiological conditions under which the one or more markers can be released include, but are not limited to, lung tissue pH, lung chemical environment, lung tissue protein expression, lung bacterial occupation, or lung viral occupation, or a combination thereof. The one or more physiological conditions can further include a blood condition or blood chemistry condition. The one or more lung tissue conditions can be associated with a disease, a condition, an infection and/or a susceptibility to a disease, a condition, or an infection. Examples of diseases, conditions, or infections include, but are not limited to, an inflammatory disease (e.g., asthma), a chronic condition (e.g., chronic obstructive pulmonary disease), a malignancy (e.g., lung cancer), overexposure to environmental pollutants (e.g., air pollution, asbestos), bacterial infection (e.g., tuberculosis), viral infection (e.g., influenza), and fungal infection (e.g., aspergillus). The method can further include providing one or more sensors configured to detect the one or more markers in the lung exhalant. The one or more sensors can be in communication with one or more controllers. The one or more controllers can be configured to regulate a time period for detecting the one or more markers in the lung exhalant from the vertebrate subject. The time period for detecting can be substantially immediately upon sensing the exhalant or delayed upon sensing the exhalant. The time period for detecting can be a predetermined time after the administration of the at least one microparticle. The one or more controllers can be configured to activate at least one chemical trigger or physical trigger to expose the one or more markers within the at least one microparticle for detection by the one or more sensors. A device is described herein that includes a device including an applicator configured to deliver at least one microparticle to lungs of a vertebrate subject, wherein the at least one microparticle includes one or more markers; and wherein the one or more markers is configured to be released in response to one or more physiological conditions in the vertebrate subject; and one or more sensors configured to detect the one or more markers in a lung exhalant of the vertebrate subject.

The one or more microparticles described herein can be configured to release one or more markers in response to detecting changes in pH of lung tissue in the vertebrate subject. A number of pulmonary inflammatory diseases can be associated with acidic lung tissue pH including, but not limited to, asthma, chronic obstructive pulmonary disease (COPD), cystic fibrosis, bronchiectasis, acute respiratory distress syndrome, bacterial infection and viral infection. The pH of healthy airway lining ranges from about pH 7.5 to about pH 7.8, as measured by pH probes to the lungs and tracheobronchial-secretion measurements. These values correlate well with measurements of pH in the expired breath of healthy individuals. In an example, the mean pH observed in expired breath from healthy individuals was about 7.7+/−0.49. By comparison, lower mean pH values were observed in patients with COPD (pH 7.16+/−0.07) and bronchiectasis (pH 7.11+/−0.07). Patients with bronchiectasis that were chronically colonized by Pseudomonas aeruginosa had significantly lower pH values compared with those noncolonized with P. aeruginosa (pH 6.96+/−0.08 versus pH 7.11+/−0.07). Acidification of the lung tissue can occur prior to the clinical recognition of a pulmonary infection, e.g., ventilator-associated pneumonia or respiratory syncytial virus, suggesting that acidic lung tissue pH may increase the susceptibility to pulmonary infection. See, e.g., Vaughan, et al., Eur. Respir. J., 22:889-894, 2003; Kostikas, et al., Am. J. Respir. Crit. Care Med., 165:1364-1370, 2002; Walsh, et al., Respir. Care, 51:1125-1131, 2006; and Tate, et al., Thorax, 926-929, 2002, each of which is incorporated herein by reference.

Changes in lung tissue pH can also contribute to susceptibility to viral infection. A number of viruses are known to infect lung tissue including, but not limited to, influenza virus, rhinovirus, parainfluenza virus, coronavirus, respiratory syncytial virus, adenovirus, cytomegalovirus, or hantavirus. Several of the early events in viral infection of a host cell are pH dependent. Early pH-dependent events include, but are not limited to, binding to specific host cell receptors, penetration of the host cell, and subsequent uncoating of the viral genome. For example, an important component of influenza infectivity is the virally-associated surface glycoprotein hemagglutinin which selectively binds in a pH dependent manner to α-sialosides on glycoproteins and glycolipids associated with the outer surface of the target cells. A conformational change in hemagglutinin is necessary for infectivity and is triggered by a drop in pH below 7.0. In another example, many enveloped and nonenveloped viruses enter the host cell via receptor-mediated endocytosis and can be processed in the low pH environment (pH approximately 5.0 to 6.5) of the endosome/lysosome and released into the host cell cytoplasm. In the case of influenza virus, the low pH environment of the endosome also activates the influenza virus M2 protein ion channel to conduct protons across the viral membrane. The lowered internal virion pH is thought to weaken protein-protein interactions between the viral matrix protein (M1) and the ribonucleoprotein (RNP) core and promote viral uncoating and replication. See, e.g., Takeda et al., J. Virol. 76:1391-1399, 2002, which is incorporated herein by reference.

The one or more microparticles described herein can be configured to release one or more markers in response to detecting the presence of one or more pathogens indicative of a physiological condition in the lung of the vertebrate subject. Examples of viral, bacterial, or fungal pathogens that infect the lungs include, but are not limited to, viruses such as influenza, rhinovirus, cytomegalovirus, paramyxovirus, respiratory syncytial virus, varicella; bacteria such as Mycoplasma, Chlamydia, Rickettsia, Klebsiella, Haemophilus influenzae, Legionella, Staphylococcus, Streptococcus, Mycobacterium; and fungi such as Histoplasma, Coccidioides, Cryptococcus, Candida, Blastomyces, Aspergillus, Actinomyces, or Nocardia.

The one or more microparticles described herein can be configured to release one or more markers in response to detecting the presence of other cell types indicative of a physiological condition in the pulmonary airway of the vertebrate subject. Examples of other cell types that invade the pulmonary airways include, but are not limited to, inflammatory cells, macrophages, neutrophils, eosinophils, basophils, and lymphocytes. Inflammatory cells in the airway can be indicative of an inflammatory condition (e.g., asthma) or infection (e.g., bacterial infection). For example, increased numbers of eosinophils in the pulmonary airways can be associated with asthma, while increased numbers of neutrophils can be associated with chronic obstructive pulmonary disease. See, e.g., Brightling, Chest, 129:1344-1348, 2006, which is incorporated herein by reference. In general, neutrophils release acidic material that can lower the pH and can increase susceptibility to an infection or exacerbate an existing infection. In addition to inflammatory cells, tumor cells can also be present in the airways, either as free cells in the airway fluid or as part of a tumor mass. For example, bronchoalveolar lavage (BAL) fluid or sputum can include atypical cells indicative of lung cancer, e.g., adenocarcinoma, in either the central or peripheral airways. See, e.g., Ahrendt, et al., J. Natl. Cancer Inst. 91:332-339, 1999; Read, et al., Prim. Care Respir. J., 15:332-336, 2006, each of which is incorporated herein by reference.

The one or more microparticles described herein can be configured to release one or more markers in response to detecting the presence of other components, e.g., proteins, lipids, carbohydrates, and/or nucleic acids, which can be indicative of a physiological condition in the lung. For example, over 100 unique protein species have been identified in bronchoalveolar lavage (BAL) fluid. See, e.g., Fietta, et al., Arthritis Res. Ther. 8:R160, 2006, which is incorporated herein by reference. Some of the proteins in the BAL fluid can be released locally in the lung by inflammatory or bronchial epithelial cells and some of the proteins can be derived from serum by diffusion across the capillary-alveolar barrier. Examples include inflammatory mediators, e.g., cytokines, chemokines, tumor antigens, e.g., carcinoembryonic antigen (CEA), or serum proteins, e.g., albumin, immunoglobulins. Other compounds that can be detected in the lung include nitric oxide, an indicator of inflammation, and volatile organic compounds linked to various cancers, e.g., butane, 3-methyl tridecane, 7-methyl tridecane, 4-methyl octane, 3-methyl hexane, heptane, 2-methyl hexane, pentane, and 5-methyl decane. See, e.g., Dweik & Amman, J. Breath Res. 030301 (3 pp), 2008; Phillips, et al., Chest 123:2115-2123, 2003; Ojoo, et al., Thorax, 60:22-26, 2005; and Psathakis, et al., Chest 125:1005-1011, 2004, each of which is incorporated herein by reference.

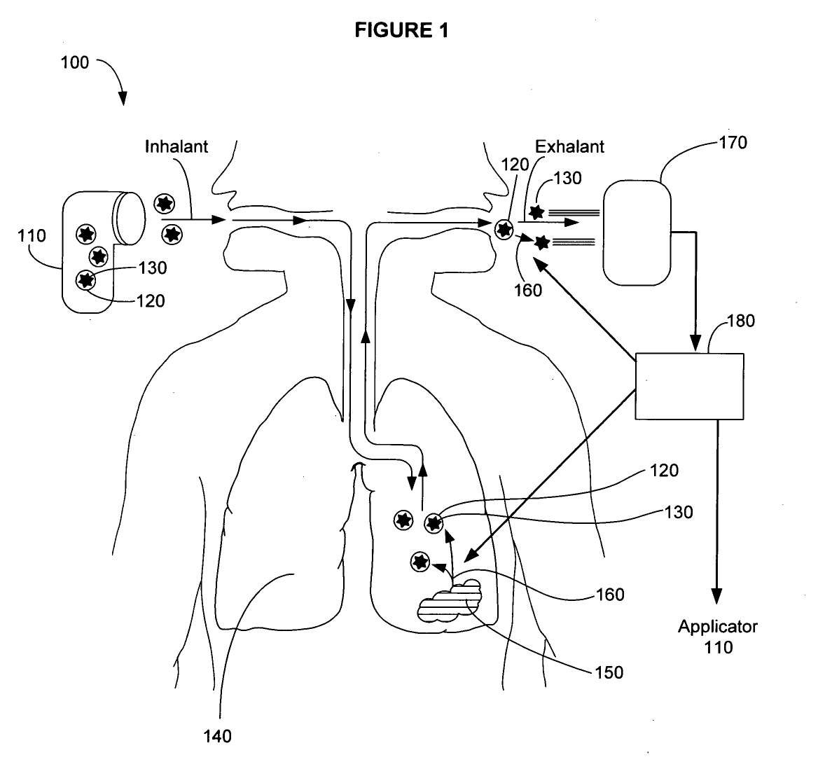

With reference to the figures, and with reference now to FIGS. 1, 2, and 3, depicted is an aspect of a device, system, or method that can serve as an illustrative environment of and/or for subject matter technologies, for example, a device is described including an applicator configured to deliver at least one microparticle to lungs of a vertebrate subject, wherein the at least one microparticle includes one or more markers; and wherein the one or more markers is configured to be released in response to one or more physiological conditions in the vertebrate subject; and one or more sensors configured to detect the one or more markers in a lung exhalant of the vertebrate subject. The specific devices, systems, and methods disclosed herein are intended as merely illustrative of their more general counterparts.

Referring to FIG. 1, depicted is a partial diagrammatic view of an illustrative embodiment of a system 100 including an applicator 110 configured to deliver at least one microparticle type 120 to lungs 140 of a vertebrate subject, wherein the at least one microparticle type includes one or more markers; and wherein the one or more markers 130 is configured to be released in response to one or more physiological conditions 150 in the vertebrate subject. The system can further include one or more sensors 170 configured to detect the one or more markers 130 in a lung exhalant of the vertebrate subject. The components of the system can be included in a single device or in multiple devices. For example, the device including the applicator 110 can be in the same device with the one or more sensors 170, or the device including the applicator 110 can be in a separate device from the one or more sensors 170. The one or more markers 130 can be, for example, embedded within the at least one microparticle 120 or within an internal compartment of the at least one microparticle 120, or can be included in a formulation or coating. Alternatively, the one or more markers 130 can be joined to the microparticle 120 by a cleavable linker. The at least one microparticle type 120 can release the one or more markers 130 based on lung tissue environment or lung cell interaction 150. At least one chemical or physical trigger 160 can be configured to expose the one or more markers 130 within the at least one microparticle type 120. In an embodiment, one or more controllers 180 can be configured to activate the at least one chemical or physical trigger 160 to release the one or more markers 130 from the at least one microparticle type 120 to the one or more sensors 170.

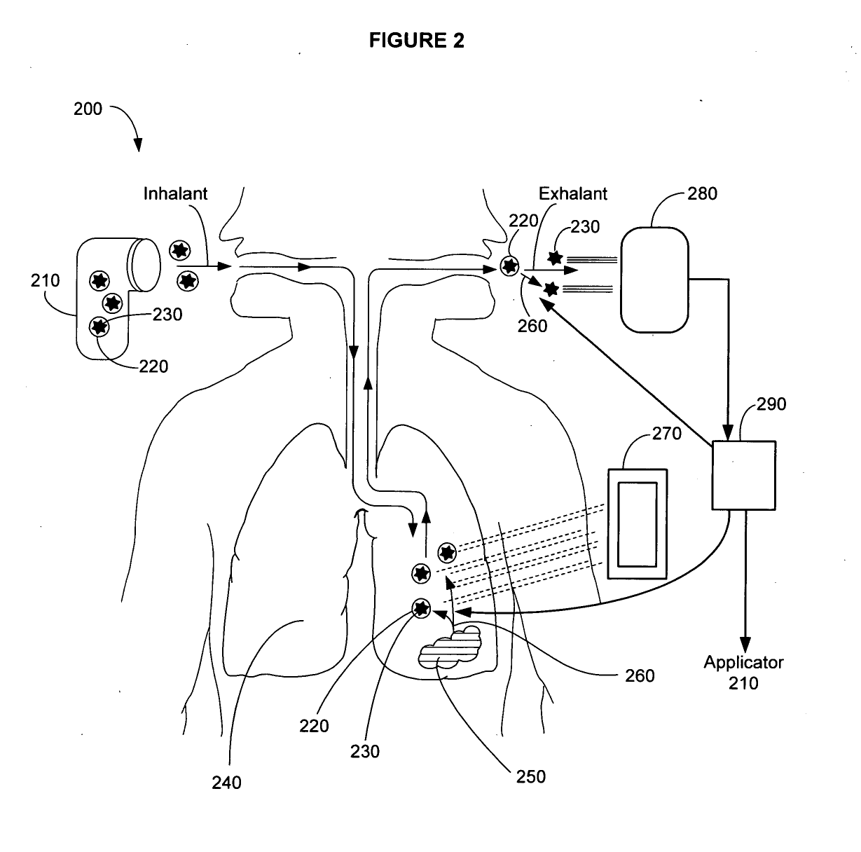

Referring to FIG. 2, depicted is a partial diagrammatic view of an illustrative embodiment of a system 200 including an applicator 210 configured to deliver at least one microparticle type 220 to lungs 240 of a vertebrate subject, wherein the at least one microparticle type is a magnetic particle 220 that includes one or more markers 230; and wherein the one or more markers 230 is configured to be released in response to one or more physiological conditions 250 in the vertebrate subject. The system can further include one or more sensors 280 configured to detect the one or more markers 230 in a lung exhalant of the vertebrate subject. One or more magnetic elements 270 can be configured to control motion of the at least one magnetic microparticle 220. The one or more markers 230 can be, for example, embedded within the at least one microparticle 220 or within an internal compartment of the at least one microparticle, or can be included in a formulation or coating. Alternatively, the one or more markers 230 can be joined to the microparticle 220 by a cleavable linker. The at least one magnetic microparticle 220 can release the one or more markers 230 based on lung tissue environment or lung cell interaction 250. At least one chemical or physical trigger 260 can be configured to release the one or more markers 230 from the at least one magnetic microparticle 220. One or more controllers 290 can be configured to activate the at least one chemical or physical trigger 260 to release the one or more markers 230 from the at least one magnetic microparticle 220 to be detected by the one or more sensors 280.

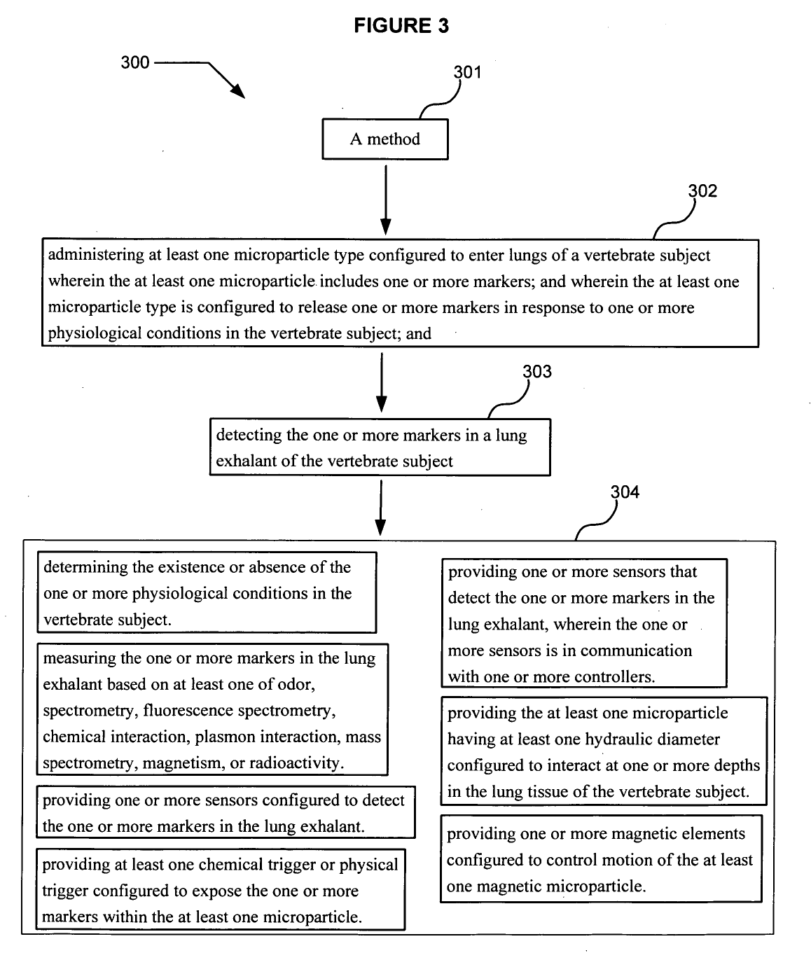

Referring to FIG. 3, depicted is a logic flowchart of a method 301 comprising administering 302 at least one microparticle to lungs of a vertebrate subject, wherein the at least one microparticle includes one or more markers; wherein the one or more markers is configured to be released in response to one or more physiological conditions in the vertebrate subject; and detecting 303 the one or more markers in a lung exhalant of the vertebrate subject.

### Stimulus-Responsive Microparticles

A method as described herein can include administering to lungs of a vertebrate subject at least one stimulus-responsive microparticle, wherein the microparticle is configured to release one or more markers in response to the stimulus, e.g., in response to a physiological condition of the vertebrate subject detected in the lung of the vertebrate subject, or an externally-applied stimulus. Examples of stimulus-responsive microparticles include, but are not limited to, pH-responsive microparticles, target-responsive microparticles, temperature-responsive microparticles, ultrasound-responsive microparticles, magnetic-responsive microparticles, and electromagnetic energy-responsive microparticles.

The one or more microparticles can include one or more pH-responsive microparticles configured to shrink or swell or hydrolyze intra-molecular bonds in response to a change in pH condition, altering the ability of the microparticles to retain an associated or encapsulated marker. pH-Responsive microparticles can include, but are not limited to, one or more pH-responsive polymers, one or more pH-responsive micelles, one or more pH-responsive liposomes, or combinations thereof. The one or more pH-responsive microparticles can be configured to release the associated or encapsulated marker at a specified pH condition in the vertebrate subject.

The pH-responsive microparticles can include one or more pH-responsive polymer. For example, microparticles composed of poly(vinylpyrrolidone-co-dimethylmaleic anhydride) retain conjugated compounds under slightly basic conditions of pH 8.5, but gradually release the compounds at neutral pH 7.0 or slightly acidic pH 6.0. See, e.g., Kamada, et al., Clin. Cancer Res. 10:2545-2550, 2004, which is incorporated herein by reference. In a further example, pH-responsive polymers can be formed using a combination of chitosan and poly-γ-glutamic acid. See, e.g., Chang, et al., Biomacromolecules, 11:133-142, 2010, which is incorporated herein by reference. Other materials configured to generate pH-responsive microparticles include, but are not limited to, diketopiperazines, biodegradable natural and synthetic polymers, proteins, polymers of mixed amino acids, alginate and poly(hydroxyl acids). See, e.g., U.S. Pat. Nos. 6,428,771; 6,071,497; 7,053,034 each of which is incorporated herein by reference.

The pH-responsive microparticles can include one or more pH-responsive micelles. For example, micelle microparticles composed of poly(ethylene glycol)-phosphatidylethanolamine can retain a fluorescent marker at pH 8.0 but rapidly release the fluorescent marker at neutral pH 7.0 and slightly acidic pH 5.0. See, e.g., Sawant, et al., Bioconjug. Chem. 17:943-949, 2006, which is incorporated herein by reference. Similarly, mixed micelles composed of poly(L-histidine)-poly(ethylene glycol) block copolymer in combination with an amphiphilic polymer are stable at neutral pH. The mixed micelles can release associated contents in acidic microenvironments. Examples of amphiphilic polymers include, but are not limited to, poly(L-lactic acid)-poly(ethylene glycol) block copolymer, poly(D,L-lactic-co-glycolic acid), poly(ethylene oxide)-poly(propylene oxide)-poly(ethylene oxide) block polymers. See, e.g., U.S. Pat. No. 7,659,314, which is incorporated herein by reference.

The pH-responsive microparticles can be composed of one or more phospholipid compounds to form pH-responsive liposomes. For example, the pH-responsive microparticles can include polymer-caged liposomes in which preformed liposomes can be treated with a cholesterol-functionalized poly(acrylic acid) additive and crosslinked. The pH-responsive microparticles including polymer-caged liposomes become highly stable and have tunable pH-sensitive responses. See, e.g., Lee, et al., J. Am. Chem. Soc. 129:15096-15097, 2007, which is incorporated herein by reference. Additional examples of pH-responsive liposomes are described in Karanth & Murthy J. Pharm. Pharmacol., 59:469-483, 2007; Auguste, et al., J. Control Release 130:266-274, 2008; U.S. Pat. Nos. 5,786,214, 5,965,434, 6,426,084, 6,897,196, 7,229,973, each of which is incorporated herein by reference.

The method as described herein can include administering one or more target-responsive microparticles, wherein the one or more microparticles are configured to release a marker in response to binding a target in the environment of the lung tissue of the vertebrate subject. Target-responsive microparticles can include one or more binding elements incorporated into the microparticles and configured to bind to a specified target. Examples of binding elements include, but are not limited to, antibodies, aptamers, oligonucleotides, protein nucleic acids, receptors, ligands, lectins, synthetic binding moieties, molecular imprinted moieties, or combinations thereof. Binding of the specified target to the microparticles alters the properties of the microparticle and allows for release of the marker. For example, target-responsive microparticles can include a target-specific aptamer, two additional overlapping oligonucleotides linked to polymerized acrylamide, and an encapsulated material. Binding of a target to the target-specific aptamer disrupts the interaction of the overlapping oligonucleotides causing aggregates of polymerized acrylamide to separate from one another and allows release of the encapsulated material. See, e.g., Yang et al., J. Am. Chem. Soc., 130:6320-6321, 2008; and Gu, et al., Proc. Natl. Acad. Sci., USA, 105:2586-2591, 2008, each of which is incorporated herein by reference. In another example, tumor-marker responsive hydrogels can be prepared by molecular imprinting. In molecular imprinting, ligands, e.g., lectins and/or antibodies, reactive with a tumor-marker can be conjugated with acrylate and polymerized with acrylamide to form the tumor-marker responsive hydrogels. See, e.g., Miyata, Proc. Natl. Acad. Sci., USA, 103:1190-1193, 2006, which is incorporated herein by reference.

The method as described herein can include administering one or more temperature-responsive microparticles, wherein the one or more microparticles are configured to release a marker in response to changes in body temperature in the vertebrate subject. The change in body temperature can include elevated endogenous temperature of the subject either globally due to a fever or locally due to inflammation, ischemia, or neoplastic tissue. The change in temperature can also include application of an external energy source to the lung to induce a localized increase in temperature. Temperature-responsive microparticles can include thermally sensitive lipid-based and/or polymer-based micelles. The micelles can be configured to encapsulate one or more marker and remain stable until a lower critical solution temperature (LCST) has been reached. For example, micelles fabricated from poly(N-isopropylacrylamide-co-N,N-dimethylacrylamide)-b-poly(D,L-lactide-co-glycolide) are stable at 37° C. but begin to release their contents at a LCST of 39° C. See, e.g., Liu, et al., Mol. Bio Syst., 1:158-165, 2005, which is incorporated herein by reference. Temperature-responsive micelles can also be composed of N-(2-hydroxypropyl) methyl acrylamide (lactate) and optionally polyethylene glycol. See, e.g., U.S. Pat. No. 7,425,581, which is incorporated herein by reference. Examples of other polymers for use in generating temperature-responsive microparticles include, but are not limited to, poly(N-(3-ethoxypropyl)acrylamide), dimethylaminoethyl methacrylate, ethylene glycol dimethacrylate, and N-isopropyl acrylamide. See, e.g., U.S. Pat. No. 6,451,429, which is incorporated herein by reference.

The method as described herein can include administering one or more stimulus-responsive microparticles, wherein the one or more microparticles are triggered to release a marker by external application of one or more transdermal energy deposition, examples of which include, but are not limited to, one or more of laser energy waves, shock waves, ultrasound waves, infrared and near infrared light, magnetic energy waves, radiofrequency energy waves and microwave waves. See, e.g., U.S. Pat. No. 6,567,257, which is incorporated herein by reference.

The method as described herein can include administering ultrasound-responsive microparticles configured to release a marker in response to a trigger, such as focused ultrasound energy. The ultrasound-responsive microparticles can include microbubbles. Microbubbles consist of a gas core surrounded by a stabilizer shell and can be configured to release their contents in response to focused ultrasound energy. The stabilizer shell can be formed from lipids/surfactants, biocompatible polymers, proteins, or combinations thereof. For example, protein-based microbubbles can be manufactured by dispersing a gas phase through a protein solution. During exposure of the protein to the newly forming gas-liquid interface, the protein is deposited on the interface and stabilizes it. The microbubbles can further include a targeting molecule that specifically directs the microbubble to a specific target location or cell type. Non-covalent streptavidin-biotin bridges or covalent crosslinking chemistries, e.g., aldehyde-amine or thiol-maleimide chemistries, can be used to incorporate a targeting molecule, e.g., antibody or aptamer, to the surface of the microbubble. See, e.g., U.S. Patent Applications 2009/0098168 and 2009/0191244; Klibanov, Med. Biol. Eng. Comput. 47:875-882, 2009; Pitt, et al., Expert Opin. Drug Deliv. 1:37-56, 2004, each of which is incorporated herein by reference.

The method as described herein can include administering magnetically-responsive microparticles, wherein the one or more microparticles are configured to contain magnetic material and accumulate in a specific target location in response to a localized magnetic field. Magnetically-responsive microparticles can be liposomes into which magnetic nanoparticles, e.g., superparamagnetic iron oxide, have been incorporated. Superparamagnetic iron oxide (SPIO; e.g., Feridex® ferumoxides; Berlex Laboratories, Montville, N.J.) and other magnetic particles can be entrapped in the core of the liposomes during liposome formation. For example, a sonication and extrusion method can be used to prepare phospholipid/polyethylene glycol/cholesterol liposomes containing SPIO. See, e.g., Kato & Artemov Magn. Reson. Med. 61:1059-1065, 2009, which is incorporated herein by reference. Similarly, lipid film evaporation and extrusion can be used to prepare phospholipid/polyethylene glycol liposomes containing ferromagnetic (magnemite) nanocrystals. See, e.g., Fortin-Ripoche, et al., Radiology, 239:415-424, 2006, which is incorporated herein by reference. The magnetically-responsive microparticles can be accumulated in a specific location, e.g., a tumor site, using a localized magnetic field. Magnetic-responsive liposomes can be further functionalized with an antibody or other binding component to increase targeting to a specific cell type, e.g., a tumor cell or an inflammatory cell. See, e.g., Ito, et al., J. Biosci. Bioeng. 100:1-11, 2005; Dames, et al., Nature Nanotech., 2:495-499, 2007; and Yang, et al., Nanomedicine 4:318-329, 2008, each of which is incorporated herein by reference.

Magnetically-responsive microparticles can include polymer-based microparticles into which a magnetic material has been incorporated. For example, magnetically-responsive microspheres can be generated using the biocompatible polymer poly D,L-lactide-glycolide (PLGA) and magnetite. PLGA magnetically-responsive microspheres can be prepared using a double emulsion solvent evaporation technique to form the PLGA microparticles followed by the addition of magnetite coated with polyethylene glycol. See, e.g., Zhao, et al., Biomagn. Res. Technol. 5: 2, 2007; Asmatulu, et al., J. Nanotechnol. Vol. 2009, Article ID 238536, 6 pages; and Cheng, et al., Parma, each of which is incorporated herein by reference. In some aspects, the polymer-based magnetically responsive microparticles can be sufficiently small enough to be incorporated into a larger liposome structure, the latter of which can be responsive to a lung condition, e.g., a pH condition of the lung.

Magnetically-responsive microparticles can be induced to accumulate in a specific location by application of a focused or localized magnetic field. An external magnet with a magnetic field of 0.3 T and a field gradient of 11 T/m is able to cause accumulation of magnetic microparticles to a specific body location in an animal subject. See, e.g., Fortin-Ripoche, et al., Radiology, 239:415-424, 2006, which is incorporated herein by reference. Microparticles containing magnetic material, e.g., superparamagnetic iron oxide, can be monitored in the lung using magnetopneumography. See, e.g., Möller, et al., J. Appl. Physiol. 97:2200-2206, 2004, which is incorporated herein by reference. Alternatively, inhaled superparamagnetic iron oxide nanoparticles can be monitored using magnetic resonance imaging. See, e.g., Martin, et al., J. Aerosol Med. Pulm. Drug Deliv. 21:335-341, 2008, which is incorporated herein by reference.

An external controller, e.g., a computer or other interactive control system, can be used to activate the transdermal energy source configured to trigger release of one or more markers from the microparticles. The external controller can control the duration of activation, the intensity of activation, and the focus of activation. The external controller can be associated with a device configured to release a transdermal energy including, but not limited to, an ultrasound device, a magnetic resonance imaging device, other magnetic energy sources, a laser or other electromagnetic energy source.

### Materials for Forming Microparticles

The microparticles configured to be administered to lungs of a subject, that are for use in the methods, devices and systems described herein, can include microspheres (uniform spheres), microcapsules (having a core and an outer layer), and particles of irregular shape. The microparticles can be polymer-based, liposome-based, or a combination thereof. Polymer-based microparticles can include particles composed of natural, synthetic, and/or bioadhesive polymers into which a marker can be incorporated. The polymer-based microparticles, e.g., hydrogels, can shrink, swell, and/or dissolve in response to a stimulus, allowing for release of the associated marker indicative of a physiological condition in the vertebrate subject. Liposome-based microparticles can be configured as vesicles composed of a phospholipid bilayer surrounding an encapsulated core into which a marker can be incorporated. The liposome-based microparticles can also shrink, swell, become leaky, or burst in response to a stimulus indicative of a physiological condition in the vertebrate subject. The microparticles can also include porous nanoparticle-aggregate particles in which nanoparticles can be formed into larger porous particles structures.

Polymer-based microparticles can be composed of natural polymers, synthetic polymers, bioadhesive polymers, or a combination thereof. Representative natural polymers for use in forming microparticles include, but are not limited to, alginate and other polysaccharides including dextran and cellulose, collagen, albumin and other hydrophilic proteins, zein and other prolamines and hydrophobic proteins, copolymers and mixtures thereof. Representative synthetic polymers for use in forming microparticles include, but are not limited to, diketopiperazines, poly(hydroxy acids) such as poly(lactic acid), poly(glycolic acid) and copolymers thereof, polyanhydrides, polyesters such as polyorthoesters, polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl ethers, polyvinyl esters, polyvinyl halides, polyvinylpyrrolidone, polyvinylacetate, and poly vinyl chloride, polystyrene, polysiloxanes, polymers of acrylic and methacrylic acids including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, poly(butic acid), poly(valeric acid), and poly(lactide-co-caprolactone). Representative bioadhesive polymers include, but are not limited to, polyhyaluronic acids, casein, gelatin, glutin, polyanhydrides, polyacrylic acid, alginate, chitosan, and polyacrylates and other bioerodible hydrogels as described by Sawhney et al., Macromolecules, 26:581-587, 1993, which is incorporated herein by reference.

Micelle-based microparticles can be formed from one or more of a variety of polymers including, but not limited to, poly(L-histidine), polyethylene glycol, poly(L-lactic acid), poly(N-vinyl-2-pyrolidone), poly(hydroxyethyl acrylate), poly(hydroxylethyl methacrylate), poly(N-(3-ethoxypropyl)acrylamide), dimethylaminoethyl methacrylate, ethylene glycol dimethacrylate, N-isopropyl acrylamide, and derivatives thereof. See, e.g., U.S. Pat. Nos. 6,451,429; 7,659,314; 7,638,558; each of which is incorporated herein by reference.

Liposome-based microparticles can be configured as vesicles composed of at least one lipid bilayer formed from one or more lipid derivative. Dried lipids placed into an aqueous environment will spontaneously associate into multilamellar structures that function as permeability barriers. These lipid vesicles or liposomes include aqueous compartments separated from each other and the external medium by a series of closed concentric lipid bilayers. The composition of the aqueous compartments is the same as the medium in which the liposomes were formed; this makes it possible to entrap a wide variety of materials within the lipid bilayers.

The liposome-based microparticles can be composed of one or more of a variety of lipids. Suitable lipids include, but are not limited to, cholesterols, phosphatidylcholines, phosphatidylethanolamines, phosphatidylserines, phosphatidylglycerols, phosphatidic acids, phosphatidylinositols, phospholipids, lysolipids, fatty acids, sphingomyelin, glycosphingolipids, glucolipids, glycolipids, sulphatides, lipids with ether and ester-linked fatty acids and polymerizable lipids, and combinations thereof. The phospholipids for use in liposomes can include phosphatidylcholines, phosphatidylethanolamines, phosphatidylserines, phosphatidylglycerols, phosphatidic acids, phosphatidylinositols, diacetyl phosphates. Fatty acids for use in liposome can include linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. Glycolipids can include cerebrosides, gangliosides, e.g., monosialoganglioside and GM1, and ceramides, e.g., lactosylceramide. Other bilayer-forming materials that can be used including long-chain dialkyl dimethyl ammonium compounds, for example, di-stearyl dimethyl ammonium compounds such as di-stearyl dimethyl ammonium chloride, di-tallow dimethyl ammonium compounds such as di-tallow dimethyl ammonium chloride and mono- and dialkyl polyoxyethylene derivatives. Either a single phospholipid or a mixture of phospholipids can be used. Sterols, for example, cholesterol or ergosterol, can be added to increase the stability of the liposomal bilayers and lipids possessing a positive or negative change. Phosphatidylethanolamine, gangliosides or phosphatic acid can be used to render the appropriate charge to the liposome and to increase the size of the aqueous compartments. Mixtures of lipids can be used to render the liposomes more fluid or more rigid and to increase or decrease permeability characteristics.

The liposome-based microparticles can be prepared by a variety of methods. These procedures have in common the dispersal of a phospholipid or mixture of lipids into a suitable container and the removal of an organic solvent, for example, ether, chloroform, or T-butanol. The organic solvent can be removed by methods such as evaporation, rotary evaporation under vacuum or lyophilization with commercially available freeze-drying equipment. Dispersing the resulting lipid film of dry lipid powder in an aqueous medium, for example, distilled water, isotonic saline or buffered solutions will result in the formation of liposomes. The one or more marker can be included in the aqueous medium during the formation of the liposomes to encapsulate the one or more marker within the forming liposomes.

The microparticles for use in the methods and systems described herein can include one or more porous nanoparticle-aggregate particles (PNAP). PNAP can include aggregates of small nanoparticles of a size compatible with efficient lung deposition (e.g., 1-5 μm), but are readily disaggregated upon deposition. See, e.g., Tsapis, et al., Proc. Natl. Acad. Sci., USA, 99:12001-12005, 2002; Edwards, et al., Science, 276:1868-1871, 1997, each of which is incorporated herein by reference.

The microparticles for use in the methods, devices, and systems described herein can include one or more gas-filled microbubbles. Microbubbles can be generally composed of a shell of biocompatible materials, e.g., proteins, lipids, or polymers, encapsulating a filling gas. The microbubble shell can be stiff (denatured proteins or polymers) or flexible (phospholipids). The shell itself can have a thickness ranging from about 10 nm to about 200 nm, with thinner shells typically used for protein and lipid microbubbles and thicker shells can be used for polymeric microbubbles. See, e.g., Chow et al., Magn. Reson. Med., 63:224-229, 2010, which is incorporated herein by reference. The microbubbles oscillate linearly when ultrasound-insonified at low acoustic amplitudes, whereas the microbubbles demonstrate nonlinear oscillating behavior at higher amplitudes. At high acoustic amplitudes, the microbubbles exhibit nonlinear behavior such as fragmentation, contents release, and jetting (see, e.g., Postema, et al., Lett. Drug Design Disc. 4:74-77, 2007, which is incorporated herein by reference).

The microparticles for use in the methods, devices, and systems described herein can include microparticles with multiple layers or walls, each layer or wall of which is capable of controlled release. For example, the microparticles can include multi-walled polymeric microcapsules. See, e.g., U.S. Pat. No. 4,861,627, which is incorporated herein by reference. For example, two polymer types can be dissolved separately in organic solvent, the solutions combined in an aqueous medium, and the organic solvent evaporated to leave two, well-defined layers in which one polymer type engulfs the other. Each layer of the multi-layer microparticle can have different dissolution properties and/or stimulus-response properties. Examples of biodegradable polymers for use in generating multi-walled microparticles include, but are not limited to, polyanhydrides, polylactic acid, polyorthoesters, and ethylene vinyl acetate. In another example, the microparticles can include multi-walled lipid vesicles. See, e.g., U.S. Pat. No. 5,853,755, which is incorporated herein by reference.

In some aspects, the microparticles can be composed of a combination of polymer, protein, and/or lipid components. For example, liposomes formed as described herein can be further coated with one or more polymer, including, but not limited to, polyethylene glycol, polylactic, polyglycolic, polylactic-polyglycolic acid copolymer, and polyvinyl alcohol. See, e.g., U.S. Pat. No. 7,368,254, which is incorporated herein by reference. Similarly, liposomes can be constructed to encapsulate one or more polymer-based nanoparticles. See, e.g., Chang, et al., Biomacromolecules 11:133-142, 2010, which is incorporated herein by reference.

The method including administering at least one microparticle to lungs of a vertebrate subject, wherein the at least one microparticle includes one or more markers; wherein the one or more markers is configured to be released in response to one or more physiological conditions in the vertebrate subject, can include administering one or more particles sizes of the same or different composition for delivery to the same or different levels of the pulmonary tissue. The one or more particle sizes can range in diameter, for example, from approximately 0.2 to 1 μm, approximately 1 to 4 μM, approximately 5 to 15 μm, approximately 15 to 40 μm, or approximately 50 to 100 μm. The size of the microparticles is an important variable in defining the deposition and the distribution of the microparticles in the pulmonary airway. See, e.g., Labiris & Dolovich, Br. J. Clin. Pharmacol. 56:588-599, 2003, which is incorporated herein by reference. Fine particles can be more readily distribute in the peripheral airways while larger particles deposit in the central airways or upper respiratory tract. A particle size can be defined by its mass median aerodynamic diameter (MMAD). Particles can be deposited by inertial impaction, gravitational sedimentation or diffusion depending upon their size. While deposition occurs throughout the airways, inertial impaction generally occurs in the first 10 generations of the lung where the air velocity is high and flow is turbulent. Deposition by gravitational sedimentation predominates in the last five to six generations of the airways (smaller bronchi and bronchioles) where air velocity is low. In the alveoli region, air velocity is negligible, and particles can be deposited by sedimentation and diffusion. Those particles not deposited during inhalation can be exhaled.

In general, larger microparticles do not readily follow changes in air flow direction and tend to deposit by inertial impaction in the upper respiratory tract. For example, most particles greater than 10 μm can be deposited in the oropharyngeal region with a large amount impacting on the larynx. Aerosols with mass median aerodynamic diameter of 5-10 μm can be mainly deposited in the large conducting airways as well as in the oropharyngeal region. Intermediate sized particles (3-5 μm) can be carried farther into the small airways of the bronchi and bronchioles, with 50% of 3 μm particles reaching the alveolar region. Particles that are less than 3 μm can behave more like gas molecules following the airflow all the way to the alveoli. However, very small particles of less the 0.5 μm, for example, may fail to be deposited in the alveoli and instead may be exhaled.

Deposition of the microparticles in the lungs can also be controlled by the inspiratory flow rate, the tidal volume and respiratory frequency of the subject. See, e.g., Labiris & Dolovich, Br. J. Cli. Pharmacol. 56:600-612, 2003, which is incorporated herein by reference. Controlling the air velocity or inspiratory flow rate by slow inhalation will maximize the number of particles that reach the alveoli and minimize the number that are exhaled. For example, fast inhalations can result in reduced peripheral deposition because the aerosol in more readily deposited by inertial impaction in the conducting airway and oropharyngeal region. When aerosols are inhaled slowly, deposition by gravitational sedimentation in peripheral region is enhanced. Peripheral deposition can also be increased with an increased in tidal volume and a decrease in respiratory frequency. As such, holding one's breath after inhalation may enable better penetration of composition into periphery of lungs.

The particle size and deposition depth of the microparticles entering the lungs can also be manipulated by the choice of the inhaler device and formulation of the microparticles. Inhalers and nebulizers of different types each have the ability to generate aerosol particles of a certain size range. For liquid formulations containing the microparticles, the size of the aerosol particle is largely a function of the design and operation of the delivery device such as the nebulizer or “atomizer” that converts the liquid into a vapor or mist. Formulating microparticles as a dry powder for inhalation, for example, can involve either micronization via jet milling, precipitation, freeze-drying or spray-drying using various excipients, such as lipids and polymers, or carrier systems such as lactose or other sugars. Particles of different sizes can be generated by modifications to the methods described above.

The size of the microparticles can be measured using any of a number of methods including, but not limited to, light scattering, x-ray sedimentation, electrical sensing using the Coulter principle, sieves, spectroscopy, and microscopy combined with image analysis. For example, microscopy with an optical microscope, a scanning electron microscope, a laser scanning microscope, a confocal microscope or a scanning probe microscope can be combined with image analysis software to determine the size and shape of particles. See, e.g., U.S. Pat. No. 7,009,169, which is incorporated herein by reference. The Clemex Particle Size Analyzer—PS3 is an example of a commercially available instrument for measuring particle size and shape using microscopy and image analysis (from Clemex Technologies, Inc., Longueuil, Canada). Another common method for particle size determination is to use a light scattering instrument, which measures the average particle size of a population of particles as well as the distribution of the particle size of the particles. When light strikes particles, scattering (diffraction) occurs. The light scatters in all directions, but for larger particles there is relatively more scattering to the front while for smaller particles there is relatively more scattering to the sides and back. The light scattering method reports a three-dimensional (i.e., volume) equivalent sphere diameter. One example of a commonly used light scattering instrument is the Horiba LA-920 laser light diffraction instrument (from Horiba Instruments, Inc., Irvine, Calif.). The light scattering method is particularly adapted to measuring particle size and particle size distributions of the small particles in a dispersion.

One or more microparticles can be sized to generate a monodisperse population of particles. Particles that are dry powder polydisperse powder particles, for example, can be sized using a series of individual and or nested sieves that can further contain beads, disks and/or other non-geometric shapes that can be rotated, vibrated or agitation in any of a number of directions to generate monodisperse particles (see, e.g., U.S. Pat. No. 6,267,310, which is incorporated herein by reference). The monodisperse population can be characterized using the particle size analysis methods described above.

In some aspects, the method includes microparticles of varying particle size and incorporating distinct markers in each particle size to enable assessment of one or more lung condition at various depths within the lung. For example, two or more pH-responsive microparticle types can be appropriately sized to assess pH at various depths within the lung. Similarly, two or more target-responsive microparticle types can be configured to assess the presence of a tumor at a specific depth in the lung.

### Markers Released in Response to One or More Physiological Conditions in the Vertebrate Subject

The method includes administering at least one microparticle to lungs of a vertebrate subject, wherein the at least one microparticle includes one or more markers; wherein the one or more markers is configured to be released in response to one or more physiological conditions in the vertebrate subject; and detecting the one or more markers in a lung exhalant of the vertebrate subject. The one or more markers can be incorporated into the microparticles and can include one or more of a gas, a vapor-phase chemical, a gas-entrained aerosol, a gas-entrained chemical, or a gas entrained nanoparticle.

The one or more markers can include one or more compositions of a gas. A gas composition can either be generated by a chemical reaction in response to a lung tissue condition or can be released from a microparticle in response to a lung tissue condition. A gas can be incorporated into a microbubble-based microparticle. Examples of gases that can be incorporated into microbubble-based microparticles include, but are not limited to, oxygen, nitric oxide, CO₂, helium, perfluoropropane, perfluorobutane, sulfur hexafluoride, and perfluorohexane vapor (see, e.g., Nishiharu, et al., Radiology 206:767-771, 1998; Postema, et al., Lett. Drug Design Disc. 4:74-77, 2007; and Klibanov, Med. Biol. Eng. Comput. 47:875-882, 2009, each of which is incorporated herein by reference).

The one or more markers can include one or more of a vapor-phase chemical. A vapor-phase chemical is a chemical with high enough vapor pressure under normal conditions (e.g., temperature in human lungs of approximately 37° C.) to significantly vaporize and enter the lung exhalant. A general example of vapor-phase chemicals can include volatile organic compounds (VOCs) which can be emitted as gases from certain solids or liquids under appropriate conditions. More specific examples of vapor-phase chemicals can include compounds used to generate a fragrance or a flavoring and can include, but not limited to, dimethyl sulfoxide (DMSO), acetaldehyde, acetophenone, trans-anethole (1-methoxy-4-propenyl benzene) (anise), benzaldehyde (benzoic aldehyde), benzyl alcohol, benzyl cinnamate, cadinene, camphene, camphor, cinnamaldehyde (3-phenylpropenal), garlic, citronellal, cresol, cyclohexane, eucalyptol, and eugenol, eugenyl methyl ether; butyl isobutyrate (n-butyl 2, methyl propanoate) (pineapple); citral (2-trans-3,7-dimethyl-2,6-actadiene-1-al); menthol (1-methyl-4-isopropylcyclohexane-3-ol); vanillin; and α-Pinene (2,6,6-trimethylbicyclo-(3,1,1)-2-heptene).

Additional examples of markers include one or more of an additive deemed “generally recognized as safe” (GRAS) by the U.S. Food and Drug Administration Center for Food Safety and Applied Nutrition, including, but not limited to, dibenzyl ether, difurfuryl ether, ethylene glycol monobutyl ether, furfuryl methyl ether, isoeugenyl benzyl ether, isocugenyl ethyl ether, methyl phenethyl ether, eta-naphthyl isobutyl ether, vanillyl butyl ether, dimethylethanolamine, isopentylideneiso-pentylamine. Additional GRAS compounds that can be readily detectable in exhaled breath include, but are not limited to, sodium bisulfate, dioctyl sodium sulfosuccinate, polyglycerol polyricinoleic acid, calcium casein peptone-calcium phosphate, botanicals (i.e., chrysanthemum; licorice; jellywort, honeysuckle; lophatherum, mulberry leaf; selfheal; sophora flower bud), ferrous bisglycinate chelate, seaweed-derived calcium, DHASCO (docosahexaenoic acid-rich single-cell oil) and ARASCO (arachidonic acid-rich single-cell oil), fructooligosaccharide, trehalose, gamma cyclodextrin, phytosterol esters, gum arabic, potassium bisulfate, stearyl alcohol, erythritol, D-tagatose, and mycoprotein.

The one or more markers can include gas-entrained aerosols, chemicals or nanoparticles or aerosols, chemicals, or nanoparticles that can be released by the microparticles and carried along by the flow of exhaled breath. For example, the marker can be deuterated water or other form of labeled water (e.g., tritiated water, (³H)₂O, or H₂¹⁸O) which when released from the microparticles becomes part of the water vapor associated with the exhaled breath. Deuterated water (D₂O) can be assessed in exhaled breath using flowing afterglow-mass spectrometry or a tunable difference frequency generation (DFG) laser source. See, e.g., Smith, et al., Am. J. Nutr. 76:1295-1301, 2002 and Wang & Sahay, Sensors 9:8230-8262, 2009, each of which is incorporated herein by reference.

The one or more markers can include one or more imaging agents including metals, radioactive isotopes, radiopaque agents, and fluorescent dyes. Radioisotopes and radiopaque agents include gadolinium, gallium, technetium, indium, strontium, iodine, barium, and phosphorus. Other radioisotopes used in medical imaging include, but are not limited to, carbon-11; nitrogen-13; oxygen-15; and fluorine-18; salts of radioisotopes such as I-131 sodium iodide, Tl-201 thallous chloride, Sr-89 strontium chloride; technetium Tc-99m; compounds containing iodine-123, iodine-124, iodine-125, and iodine-131; compounds containing indium-111 such as ¹¹¹In-1,4,7,10-Tetraazacyclododecane-N,N′,N″,N″-tetraacetic acid and ¹¹¹In-Diethylenetriamine pentaacetic acid; ¹⁷⁷Lu-[(R)-2-amino-3-(4-isothiocyanatophenyl)propyl]-trans-(S,S)-cyclohexane-1,2-diamine-pentaacetic acid) (¹⁷⁷Lu-CHX-A″-DTPA), ⁶⁴Cu-DOTA, ⁸⁹Zr, and ⁸⁶Y-DOTA.

The one or more markers can include one or more fluorescent dyes commonly used for diagnostic fluorescence imaging including fluorescein (FITC), indocyanine green (ICG) and rhodamine B. Examples of other fluorescent dyes for use in fluorescence imaging include, but are not limited to, cyanine dyes such as Cy5, Cy5.5, and Cy7 (Amersham Biosciences, Piscataway, N.J., USA) and/or Alexa Fluor dyes such as Alexa Fluor 633, Alexa Fluor 635, Alexa Fluor 647, Alexa Fluor 660, Alexa Fluor 680, Alexa Fluor 700 and Alexa Fluor 750 (Molecular Probes-Invitrogen, Carlsbad, Calif., USA; see, e.g., U.S. Pat. App. No. 2005/0171434, incorporated herein by reference). Additional fluorophores include IRDye800, IRDye700, and IRDye680 (LI-COR, Lincoln, Nebr., USA), NIR-1 and 1C5-OSu (Dojindo, Kumamoto, Japan), La Jolla Blue (Diatron, Miami, Fla., USA), FAR-Blue, FAR-Green One, and FAR-Green Two (Innosense, Giacosa, Italy), ADS 790-NS and ADS 821-NS (American Dye Source, Montreal, Calif.), NIAD-4 (ICx Technologies, Arlington, Va.). Other fluorescing agents include BODIPY-FL, europium, green, yellow and red fluorescent proteins, and luciferase. Quantum dots of various emission/excitation properties are also available for fluorescence detection. See, e.g., Jaiswal et al., Nature Biotech. 2003, 21:47-51, which is incorporated herein by reference. The fluorescent dye is preferably attached to a larger molecule or particle to more readily facilitate exhalation.

### Delivery Device

A delivery device including an applicator can be used to deliver at least one microparticle to lungs of a vertebrate subject, wherein the at least one microparticle includes one or more markers; and wherein the one or more markers is configured to be released in response to one or more physiological conditions in the vertebrate subject. The microparticles can be administered to a subject by liquid aerosolization as a suspension or in an appropriate aqueous medium. Alternatively, the microparticles can be dried and micronized to one or more appropriate particle size either with or without added excipients and administered by dry powder inhalation. Examples of inhaling devices include, but are not limited to, inhalers, atomizers, aerosolizers, misters, metered-dose inhalers, metered-dose misters, metered dose sprayers, metered dose atomizer, dry powder inhalers, and nebulizers. The pressurized metered-dose inhaler commonly includes a solution in a pressurized canister attached to a hand-operated actuator. On activation, the metered-dose inhaler releases a fixed dose of microparticles in aerosol form that can be inhaled by the subject. In contrast, dry powder inhalers deliver the microparticles in a dry form. Dry powder inhalers can be configured to rely on the inhalation by the subject to entrain the powder from the device and subsequently breakup the particles into sufficiently small particles to reach the depths of the lungs. Nebulizers deliver microparticles in the form of a mist or aerosol. The aerosol can be formed from a liquid solution using compressed air, vibration of a membrane or mesh, or, ultrasonic vibration.

### Sensors Detect Markers in a Lung Exhalant

The method described herein further includes providing one or more sensors configured to detect the one or more markers in a lung exhalant indicative of the one or more lung tissue conditions in the lung exhalant of a vertebrate subject. The one or more sensors can be appropriately configured to sense a marker that is a gas, a vapor-phase chemical, a gas-entrained aerosol, a gas-entrained chemical, or a gas entrained nanoparticle, or the like and will vary depending upon the physical and chemical nature of the marker.

Devices, systems and methods for detecting markers in the lung exhalant can include gas sensor technology, such as an “artificial” or “electronic” tongue or nose, configured to non-invasively monitor the concentration of a marker in exhaled breath. Electronic noses have been used in a variety of commercial industries including agricultural, biomedical, cosmetics, environmental, food manufacturing, military, pharmaceutical, and regulatory. Common examples of electronic nose devices include evidential breath measurement devices used by law enforcement to assess alcohol levels in the exhaled breath of impaired drivers. See, e.g., Department of Transportation, Federal Register, Vol. 72, No. 241, pp. 71480-71483, Dec. 17, 2007, which is incorporated herein by reference. These devices can sense the level of alcohol by using infrared spectroscopy, by using a chemical reaction between alcohol, sulfuric acid and potassium dichromate, or by using oxidation of alcohol. For example, infrared spectroscopy assesses how well components of exhaled breath absorb infrared light. A broadband IR beam is passed through the sample chamber containing the exhaled breath and is focused by a lens onto a spinning filter wheel which contains narrow band filters specific for the wavelengths of the bonds in ethanol. The light passing through each filter is detected by the photocell, where it is converted to an electrical pulse and the electrical pulse is relayed to the microprocessor which interprets the pulses and calculates the blood alcohol content based on the absorption of infrared light.

A number of sensor types have been described for use in electronic nose devices including acoustic sensors, e.g., quartz crystal microbalance, surface acoustic wave (SAW), or bulk acoustic wave (BAW) sensors; calorimetric sensors; colorimetric sensors; electrochemical sensors; fluorescence sensors; infrared sensors; metal-oxide semi conducting sensors (MOS); conducting polymer sensors; conductive electroactive polymer sensors; and optical sensors. See, e.g., Wilson & Baietto, Sensors 9:5099-5148, 2009, which is incorporated herein by reference. The sensors in an electronic nose can be configured into arrays and are capable of defining a fingerprint for a specific marker relative to other components of the sample.

Conducting or conductive-polymer gas-sensors (also referred to as “chemoresistors”) operate based on changes in electrical resistance caused by adsorption of markers onto the sensor surface. Conductive polymer gas sensors consist of a substrate, e.g., silicon, a pair of metal plated electrodes, and a conducting organic polymer coating or film. The binding of the marker molecules to the conducting polymer film induces a change in electric resistance and the variation in this resistance enables identification of the target marker and its concentration. These types of sensors can function at or close to room temperature, have high sensitivities, short response times, can be easily synthesized, and have good mechanical properties. Different sensitivities for detecting different markers can be obtained by modifying or choosing an alternate conductive polymer. Examples of sensor coating monomers include, but are not limited to, polypyrrole, polyanaline, polythiophene, polyacetate, poly(phenylvinylene), poly(3,4-ethylenedioxythiophene), poly(N-vinylcarbonate), poly(thienylenevinylene). See, e.g., Wilson & Baietto, Sensors 9:5099-5148, 2009, which is incorporated herein by reference.

Polymeric gas sensors as well as other sensor types, can be built into an array of sensors in which each sensor is designed to respond differently to different markers. For example, a sensor can comprise of an array of polymers, (i.e., 20 different polymers) each exposed to a marker. Each of the individual polymers react differently to the presence of a marker, creating a change in the resistance of that membrane and generating an analog voltage or a “signature” in response to that specific marker. The normalized change in resistance can then be transmitted to a processor to identify the type, quantity, and quality of the marker based on the pattern change in the sensor array. The unique response results in a distinct electrical fingerprint that is used to characterize the marker. The pattern of resistance changes of the array is indicative of the marker in the sample, while the amplitude of the pattern is indicative of the concentration of the marker in the sample.

Acoustic wave gas sensors use a mechanical (acoustic) wave as the sensing mechanism. As the acoustic wave propagates through the sensor coating material (e.g., bulk acoustic wave (BAW)) or on the surface of the sensor coating material (e.g., surface acoustic wave (SAW)), any changes to the characteristics of the propagation path due to the sorption of the marker affects the velocity and/or the amplitude of the wave. Acoustic wave gas sensors commonly consist of a piezoelectric substrate such as, for example, quartz, lithium niobate, lithium tantalite, or zinc oxide, doped with a suitable marker-specific sorptive material. For example, a SAW sensor can include a piezoelectric substrate covered by a polymer coating capable of selectively absorbing a marker, and oscillating, high frequency waves propagating along the surface of the substrate between sets of interdigitated electrodes (i.e., to form a transducer). When a marker interacts with the polymer coating on the piezoelectric substrate, the interaction causes a change in the amplitude and/or velocity of the propagated wave. The detectable change in the propagated wave is generally proportional to the mass load of the marker(s), i.e., concentration of the marker in exhaled breath. See, e.g., U.S. Pat. Nos. 4,312,228; 4,895,017; and 5,325,704; and Ho, et al., Sensors, 3:236-247, 2003, each of which is incorporated herein by reference. Similar types of sensors include bulk acoustic wave (BAW) devices, plate acoustic wave devices, interdigitated microelectrode (IME) devices, optical waveguide (OW) devices, electrochemical sensors, and electrically conducting sensors.

Optical sensor systems measure light modulations in response to a marker released in an exhalant of the vertebrate subject. Optical sensor systems can include an assortment of technologies using diverse light sources, e.g., optical fibers, photodiodes, and/or light-sensitive photodetectors. Operational modes can include measured changes in absorbance, fluorescence, light polarization, optical layer thickness, or colorimetric dye response. Simple optic sensors use color changing indicators such as metalloporphyrins, to measure absorbance with a LED and photodetector system upon exposure to gas analytes. Two specialized types of optical sensors include the colorimetric and fluorescence sensors. Colorimetric sensors use thin films of chemically-responsive dyes as a colorimetric sensor array. Fluorescence sensors detect fluorescent light emissions from the marker and can be more sensitive than colorimetric sensor arrays. A fluorescence sensor, for example, can include a film comprised of a fluorescent polymer in which the fluorescence intensity of the polymer decreases in response to binding a marker. The sensor can be configured such that a single molecule binding event can quench the fluorescence of many polymer subunit, resulting in an amplification of the quenching. The polymer can be further configured such that the binding of markers to the film is reversible, enabling reuse of the sensor. See, e.g., U.S. Pat. No. 6,001,556; U.S. Patent Application 2008/0050839; Charych, et al., Science, 261:585-588, 1993; and Chen, et al., Proc. Natl. Acad. Sci., USA, 96:12287-12292, 1999, each of which is incorporated herein by reference.

A device including one or more sensors can include laser spectroscopic techniques to sense one or more markers in exhaled breath of the vertebrate subject. These techniques include, but are not limited to, tunable diode laser absorption spectroscopy, cavity ring-down spectroscopy, integrated cavity output spectroscopy, cavity enhanced absorption spectroscopy, cavity leak-out spectroscopy, photoacoustic spectroscopy, quartz-enhanced photoacoustic spectroscopy, and optical frequency comb cavity-enhanced absorption spectroscopy. Spectral fingerprints of markers using these techniques can span from the UV to the mid-IR spectral regions with detection limits ranging from parts per million to parts per billion. See, e.g., Wang & Sahay, Sensors, 9:8230-8262, 2009, which is incorporated herein by reference.

Metal-oxide gas sensors can include the most widely used class of gas sensors. A metal-oxide sensor consists of a ceramic support tube containing a heater spiral, usually composed of platinum. The ceramic is coated with a metal-oxide, a common example of which is tin-dioxide (SnO₂), and doped with small amounts of catalytic metal additives. The sorption of gas molecules causes changes in conductivity brought about by a combustion reaction with oxygen species on the surface of the tin-dioxide particles. These sensors operate at temperatures ranging from 300° C. to 550° C.

Ion mobility spectrometry (IMS) separates ionic species at atmospheric pressure based on drift time in the presence of an electrical field. The sample is drawn into the instrument and ionized by a weak radioactive source. The ionized molecules drift through the cell under the influence of an electric field. An electronic shutter grid allows periodic introduction of the ions into the drift tube where they separate based on charge, mass, and shape. Smaller ions move faster than larger ions through the drift tube and arrive at the detector sooner. The amplified current from the detector is measured as a function of time and a spectrum is generated. A microprocessor evaluates the spectrum for the target marker compound, and determines the concentration based on the peak height. IMS is a sensitive and extremely fast method of marker detection and allows for near real time analysis.

Other sensors for sensing one or more markers in exhaled breath of the vertebrate subject can include gas chromatography, liquid chromatography, mass spectrometers (including proton transfer reaction mass spectrometry), or combinations thereof; infrared (IR) or ultraviolet (UV) or visible or fluorescence spectrophotometers (i.e., non-dispersive infrared spectrometer); aptamer sensor technology; amplifying fluorescent polymer (AFP) sensor technology; photo-ionization detectors, or ion mobility spectrometry technology. See, e.g., Simon & Davis, “Instrumentation and Sensors for Human Breath Analysis,” in Advances in Biomedical Sensing, S. C. Mukhopadhyay & A. Lay-Ekuakille (Eds). Vol 55, pp. 144-165, 2010, Springer-Verlag Berlin Heidelberg, which is incorporated herein by reference. The one or more sensors can include, but are not limited to, one or more of a biosensor, a chemical sensor, a physical sensor, an optical sensor, a radioactivity sensor, or a combination thereof. The sensors can further include one or more sensors optionally small in size, for example a sensor or array that is a chemical sensor (Snow, Science 307:1942-1945, 2005), a gas sensor (Hagleitner, et al., Nature 414:293-296, 2001), an electronic nose, and/or a nuclear magnetic resonance imager (Yusa, Nature 434:1001-1005, 2005), each of which is incorporated herein by reference.

The one or more sensors can include a binding component that selectively binds one or more markers in the exhaled breath. The interaction of one or more markers with one or more binding component on the sensors results in one or more detectable signals. Examples of binding components include antibodies, oligonucleotides, aptamers, protein-nucleic acids, ligands, receptors, and synthetic binding moieties. The one or more binding component is linked to sensor. For example, an antibody or an aptamer can be incorporated into resonant oscillating quartz sensors that can detect minute changes in resonance frequencies due to modulations of mass of the oscillating system, which results from a binding or dissociation event, i.e., binding with a target marker in the exhalant.

The one or more sensors for detecting a marker in an exhalant of the vertebrate subject can be one or more microcantilevers. A microcantilever can act as a biological sensor by detecting changes in cantilever bending or vibrational frequency in response to binding of one or more markers to the surface of the sensor. In some aspects, the sensor can be bound to a microcantilever or a microbead as in an immunoaffinity binding array. In some aspects, a biochip can be formed that uses microcantilever bi-material formed from gold and silicon, as sensing elements. See, e.g. Vashist J. Nanotech Online 3:DO: 10.2240/azojono0115, 2007, which is incorporated herein by reference. The gold component of the microcantilever can be coated with one or more binding component which upon binding one or more marker causes the microcantilever to deflect. Aptamers or antibodies specific for one or more markers can be used to coat microcantilevers. See, e.g., U.S. Pat. No. 7,097,662, which is incorporated herein by reference. The one or more sensor can incorporate one or more methods for microcantilever deflection detection including, but not limited to, piezoresistive deflection, optical deflection, capacitive deflection, interferometry deflection, optical diffraction grating deflection, and charge coupled device. In some aspects, the one or more microcantilever can be a nanocantilever with nanoscale components. The one or more microcantilevers and/or nanocantilevers can be arranged into arrays. Both microcantilevers and nanocantilevers can find utility in microelectomechnical systems (MEMS) and/or nanoelectomechnical systems (NEMS).

The one or more sensors for detecting a marker in an exhalant of the vertebrate subject can be a field effect transistor (FET) based biosensor. In some aspects, a change in electrical signal is used to detect interaction of one or more markers with one or more binding components of the sensor. See, e.g., U.S. Pat. No. 7,303,875, which is incorporated herein by reference.

The one or more sensors can use Förster or fluorescence resonance energy transfer (FRET) to sense one or more markers in the exhaled breath of a subject. As described herein, FRET is a distance-dependent interaction between the electronic excited states of two dye molecules in which excitation is transferred from a donor molecule to an acceptor molecule without emission of a photon. For use in a sensor, the one or more binding components associated with the one or more sensors can include at least one donor molecule and at least one acceptor molecule. Binding of a marker to the binding component results in a conformation change in the binding component and leads to changes in the distance between the donor and acceptor molecules with associated changes in measurable fluorescence.

A variety of donor and acceptor fluorophore pairs can be considered for FRET including, but not limited to, fluorescein and tetramethylrhodamine; IAEDANS and fluorescein; fluorescein and fluorescein; and BODIPY FL and BODIPY FL, and various Alexa Fluor pairings as described herein. The cyanine dyes Cy3, Cy5, Cy5.5 and Cy7, which emit in the red and far red wavelength range (>550 nm) as well as semiconductor quantum dots can also be used for FRET-based detection systems. Quenching dyes can be used to quench the fluorescence of visible light-excited fluorophores, examples of which include DABCYL, the non-fluorescing diarylrhodamine derivative dyes QSY 7, QSY 9 and QSY 21 (Molecular Probes, Carlsbad, Calif., USA), the non-fluorescing Black Hole Quenchers BHQ0, BHQ1, BHQ2, and BHQ3 (Biosearch Technologies, Inc., Novato, Calif., USA) and Eclipse (Applera Corp., Norwalk, Conn., USA). A variety of donor fluorophore and quencher pairs can be considered for FRET associated with the target recognition element including, but not limited to, fluorescein with DABCYL; EDANS with DABCYL; or fluorescein with QSY 7 and QSY 9. In general, QSY 7 and QSY 9 dyes efficiently quench the fluorescence emission of donor dyes including blue-fluorescent coumarins, green- or orange-fluorescent dyes, and conjugates of the Texas Red and Alexa Fluor 594 dyes. QSY 21 dye efficiently quenches all red-fluorescent dyes.

The one or more sensor for detecting a marker in an exhalant of the vertebrate subject can use the technique of surface plasmon resonance (for planar surfaces) or localized surface plasmon resonance (for nanoparticles) in which changes in the refractive index can be measured on a sensor surface in response to changes in molecules bound on the sensor surface. The surface of the sensor can include a glass support or other solid support coated with a thin film of metal, for example, gold, as well as a matrix of immobilized binding components configured to recognize one or more markers. The sensor is illuminated by monochromatic light. Resonance occurs at a specific angle of incident light. The resonance angle depends on the refractive index in the vicinity of the surface, which is dependent upon the concentration of marker bound to the surface. An example of instrumentation that uses surface plasmon resonance is the BIACORE system (Biacore, Inc.—GE Healthcare, Piscataway, N.J.) which includes a sensor microchip, a laser light source emitting polarized light, an automated fluid handling system, and a diode array position sensitive detector. See, e.g., Raghavan & Bjorkman Structure 3:331-333, 1995, which is incorporated herein by reference.

The one or more sensors for detecting a marker in an exhalant of the vertebrate subject can be one or more label-free optical biosensors that incorporate other optical methodologies, e.g., interferometers, waveguides, fiber gratings, ring resonators, and photonic crystals. See, e.g., Fan, et al., Anal. Chim. Acta 620:8-26, 2008, which is incorporated herein by reference.

The one or more sensors can include a binding component that selectively binds one or more markers in the exhaled breath. The one or more binding components configured to detect the one or more markers can include one or more antibodies that bind one or more markers. Antibodies or fragments thereof for use as one or more binding components include, but are not limited to, monoclonal antibodies, polyclonal antibodies, Fab fragments of monoclonal antibodies, Fab fragments of polyclonal antibodies, Fab₂ fragments of monoclonal antibodies, and Fab₂ fragments of polyclonal antibodies, chimeric antibodies, non-human antibodies, fully human antibodies, among others. Single chain or multiple chain antigen-recognition sites can be used. Multiple chain antigen-recognition sites can be fused or unfused. Antibodies or fragments thereof can be generated using standard methods. See, e.g., Harlow & Lane (Antibodies: A Laboratory Manual, Cold Spring Harbor Laboratory Press; 1^{st} edition 1988), which is incorporated herein by reference. Alternatively, an antibody or fragment thereof directed against one or more marker can be generated, using phage display technology. See, e.g., Kupper, et al. BMC Biotechnology 5:4, 2005, which is incorporated herein by reference. An antibody, a fragment thereof, or an artificial antibody, e.g., Affibody® artificial antibodies (Affibody AB, Bromma, Sweden) can be prepared using in silico design. See, e.g., Knappik et al., J. Mol. Biol. 296: 57-86, 2000, which is incorporated herein by reference. In some aspects, antibodies directed against one or more markers can be available from a commercial source, e.g., from Novus Biological, Littleton, Colo.; Sigma-Aldrich, St. Louis, Mo.; United States Biological, Swampscott, Mass.

The one or more binding components configured to detect the one or more markers can include one or more aptamers. DNA or RNA aptamers include artificial oligonucleotides (DNA or RNA) which can bind to a wide variety of entities (e.g., metal ions, small organic molecules, proteins, and cells) with high selectivity, specificity, and affinity. Aptamers can be isolated from a large library of 10¹⁴ to 10¹⁵ random oligonucleotide sequences using an aptamer-ligand binding assay and an iterative in vitro selection procedure often termed “systematic evolution of ligands by exponential enrichment” (SELEX). See, e.g., Cao, et al., Current Proteomics 2:31-40, 2005; Proske, et al., Appl. Microbiol. Biotechnol. 69:367-374, 2005; and Jayasena, Clin. Chem. 45:1628-1650, 1999, each of which is incorporated herein by reference. Aptamers can be used with a variety of sensor systems including, but not limited to, electrochemical sensors, e.g., electrochemical impedance spectroscopy, electrostatic interactions; optical sensors, e.g., surface plasmon resonance, evanescent wave spectroscopy, fluorescence and luminescence detection; mass sensitive sensors, e.g., piezoelectric quartz microbalance), potentiometric sensors (e.g., field-effect transistors. See, e.g., Strehlitz, et al., Sensors 2008, 8:4296-4307, which is incorporated herein by reference.

The one or more sensors for sensing one or more markers in the exhalant of the vertebrate subject can be connected to an analyzer configured to compare a pattern of response to previously measured and characterized responses from known markers. A pattern recognition algorithm, e.g., neural network, can be used compare the sensor data with previous measurements, standard curves, fingerprint patterns, and the like. Examples of pattern recognition algorithms suited to use with chemical sensor array data include, but are not limited to, probabilistic neural networks, learning vector quantization neural networks, back-propagation artificial neural networks, soft independent modeling of class analogy, Bayesian linear discriminant analysis, Mahalanobis linear discriminant analysis, and the nearest-neighbor pattern recognition algorithms. See, e.g., Shaffer, et al., Analytica Chimica Acta, 384:305-317, which is incorporated herein by reference. By comparing the output from a sensor or sensor array to a “blank” or control, for example, a pattern recognition algorithm, e.g., neural network, can establish a pattern that is unique to that marker and subsequently learns to recognize that marker.

### Breath Analyzers for Detecting or Sensing Markers

A device that detects marker released from a microparticle can be indicative of a condition in the lung. The marker can be measured in the exhaled breath of the subject using one or more sensors as described herein. The one or more sensors can be associated with a device configured to sample the exhaled breath either in-line as it passes by or following collection into a separate compartment. Generally, the exhalation gas stream from the lungs comprises a series of stages. At the beginning of exhalation or initial stage of exhalation, the representative gas is coming from an anatomically inactive (dead space) part of the respiratory system, e.g., from the mouth and upper respiratory tracts. The initial stage of exhalation is followed by a plateau stage of exhalation. Early in the plateau stage of exhalation, the gas is a mixture of dead space and metabolically active gases. The last stage of exhalation comprises primarily deep lung gas or alveolar gas. This gas, which comes from the alveoli, is termed end-tidal gas. The exhaled breath sample can be collected at one or more stages in this exhalation process.

In some aspects, the exhaled breath sample is collected at the end-tidal stage. Technology similar to that used for end-tidal carbon dioxide monitoring can be used to determine when the sample is collected. See, e.g., St. John, Crit. Care Nurse, 23:83-88, 2003, which is incorporated herein by reference. Methods for airway pressure and airway flow measurements provide other means of collecting samples at the appropriate phase of the respiratory cycle. Single or multiple samples can be collected using side stream methods in which samples can be collected through an adapter at the end of a breathing tube and drawn out through tubing to a separate compartment. The separate compartment can contain one or more sensors configured to assess the presence of an exhaled marker. Depending on the sample size and sensor response time, exhaled gas can be collected in successive cycles. Under conditions of rapid sensor acquisition time, in-line sampling can be used. With in-line sampling, one or more sensors or sensor arrays can be placed at the proximal end of the breathing tube directly in the gas stream. As an alternative to sampling at the end-tidal stage, samples can be taken throughout exhalation and an average value determined.

Sampling of the exhaled breath can be done using a handheld device such as those used for analysis of blood alcohol levels and other components of exhaled breath. See, e.g., National Highway Traffic Safety Administration, Federal Register Vol. 72, No. 241, pp. 71480-71483, Dec. 17, 2007; U.S. Pat. Nos. 7,364,551; 7,417,730; each of which is incorporated herein by reference. In some aspects, the exhaled breath is collected in a handheld sample collector and subsequently connected to a second device configured with one or more sensors to sense a marker in the exhaled breath. See, e.g., U.S. Pat. No. 7,153,272, which is incorporated herein by reference. In this instance, the handheld sample collector can be connected to a larger, less portable device such as, for example, a gas chromatograph or mass spectrometer. Preferably the device configured to sense a marker in the exhalant is also capable of delivering the microparticles containing the marker through inhalation. An example of a device is provided for both delivery of an agent upon inhalation and analysis of exhalation parameters. See, e.g., U.S. Pat. No. 7,451,760, which is incorporated herein by reference.

### Communicating Results of Analysis

The method further includes providing the results of analysis of markers in the lung exhalant of the vertebrate subject. The results can be provided to the subject, to a medical provider, to a caregiver, to an authority, e.g., health department, immigration officer, school administrator, law enforcement, or to a combination thereof via a reporting device. In some aspects, the sensor technology used to measure a marker in the lung exhalant includes a device for reporting the results. The device or system for reporting the results can include a digital display panel associated with the device implementing the sensor technology. Alternatively, the device or system for reporting the results can include a computer processor linked to the sensor technology in which electronic or printed results can be provided or transportable read/write magnetic media such as a disk, a flash drive, a flash card, or other recordable and transportable recording media. The device or system for reporting the results can also include a physical printout or report generated from a thermal, laser, ink-jet, or other printer technology.

In some instances, the results of analysis of markers in the lung exhalant may prompt an immediate response. For example, positive results of a test using tuberculosis-responsive microparticles at an airport or other crowded testing location may prompt immediate quarantine of a subject to prevent spread of the infection. Similarly, positive results of a test using pH-responsive microparticles may suggest increased susceptibility to viral infection and may prompt immediate preventative action, e.g., administration of prophylactic agent or barrier protection with a mask. In some instances, the results of analysis of markers in the lung exhalant may prompt a less immediate response. For example, positive results of a test using immune cell target-responsive microparticles with a hand held unit used in a home setting may prompt a subject with a chronic inflammatory condition to seek medical attention to adjust the subject's medication, e.g., anti-inflammatory medication.

In those instances in which the results are not provided in real-time, the device for reporting the results can include providing the results to the subject via facsimile, electronic mail, mail or courier service, or any other means of safely and securely sending the report to the subject. Other devices for reporting the results can include, but are not limited to, an interactive voice response system, interactive computer-based reporting system, interactive telephone touch-tone system, or other similar system.

### PROPHETIC EXAMPLES

### Example 1

### Method for Administering Microparticles by Inhalation to a Human Subject for Monitoring a pH Condition in the Lung Indicating Presence or Increased Susceptibility of the Human Subject to Infection by Viral Particles

Changes in pH condition in the lungs relative to the normal physiological range may increase susceptibility of a human subject to infection by viral particles (e.g., influenza virus). A method and system is described for administering microparticles by inhalation to a human subject for monitoring a pH condition in the lung of the human subject. The microparticles are formulated from a biodegradable material, e.g., poly(D,L-lactic-co-glycolic acid) (PLGA). The microparticles are configured to release a marker, a pH-sensitive fluorescent cyanine dye, into the exhaled breath of a human subject when exposed to a pH condition in the lungs. The normal range of pH values of fluid lining human airways ranges from about pH 7.5 to 7.8. A pH value of 7.0 or lower can increase the susceptibility of a human subject to a viral infection. The microparticles incorporating the pH-sensitive cyanine dye may be configured to be inhaled and exhaled by the human subject. An increase in peak UV/visible light absorption maxima of particles including the pH-sensitive cyanine dye in the exhaled breath of the human subject is indicative of a more acidic pH condition in the lungs and increased susceptibility to viral infection

The PLGA microparticles are of a size that has poor lung deposition efficiency and are readily exhaled following initial inhalation. Particles of approximately 1 micron delivered as an aerosol are appropriate for this application, with poor deposition in the lungs during a single inhalation/exhalation cycle. See, e.g., Kim et al. (J. Appl. Physiol. 81:2203-2213, 1996, which is incorporated herein by reference. PLGA microparticles in the 1 micron range may be synthesized. The one micron PLGA microparticles are suitable for delivery to the lungs as an aerosol. See, e.g., Doan & Olivier Int. J. Pharm. 382:61-66, 2009, which is incorporated herein by reference. To synthesize PLGA microparticles, a coarse emulsion of PLGA in ethyl acetate is mixed with polyvinyl alcohol and subjected to homogenization under pressure. The ethyl acetate is evaporated and the resulting microparticles are collected by centrifugation, washed and dried. The size distribution of the microparticles is assessed by laser diffraction spectroscopy using a Coulter counter equipped with a polarization intensity differential scattering detection system. See, e.g., Multisizer™ 3 COULTER COUNTER®, Beckman Coulter, Fullerton, Calif. The morphology and texture of the microparticles are examined by optical microscopy and scanning electron microscopy.

The microparticles incorporate a pH-sensitive dye, e.g., a cyanine dye. The pH-sensitive cyanine dye is synthesized using conventional methods. See, e.g., Briggs et al., Chem. Comm. 2323-2324, 2000, which is incorporated herein by reference. Briefly, 1-ethyl-2,3,-trimethyl-3H-indol-1-ium-5-sulfonate and 5-carboxymethyl-2,3,3-trimethyl-2,3-dihydroindole are condensed with malonaldehyde bis(phenylimine) in the presence of acetic acid, acetic anhydride and pyridine. The cyanine dye is further modified to include an N-hydroxysuccinimidyl ester to facilitate conjugation to the microparticles.

The pH sensitive cyanine dye exhibits an absorption maximum at 645 nm when excited at an energy of 630 nm (see, e.g., Briggs et al., Chem. Comm. 2323-2324, 2000, which is incorporated herein by reference). Under conditions of increasing pH, e.g., pH 4.5 to pH 9.0, the absorption maximum at 645 decreases. When the pH sensitive cyanine dye is exposed to a broad UV/visible spectra under the conditions of increasing pH, e.g., pH 4.5 to pH 9.0, a second peak emerges at a wavelength of 480 nm. The relative difference between the absorption peaks observed at 645 nm and 480 nm are used to indicate changes in pH.

The microparticles labeled with the pH sensitive cyanine dye are incorporated into an inhaler apparatus which includes a UV/visible light energy source and an optical sensor or sensor array, e.g., charge coupled device (CCD), capable of detecting the fluorescence associated with the microparticles. The human subject inhales the microparticles and then exhales. The exhaled breath is passed back through the inhaler. The optical sensor associated with the inhaler is configured with appropriate filters to measure peak absorption maxima at 480 nm and 645 nm associated with the labeled microparticles. A comparison of the peak absorption maxima at 480 nm and at 645 nm upon exhalation of the labeled microparticles relative to the peak absorption maxima at 480 nm and at 645 nm upon inhalation of the microparticles is used to assess changes in pulmonary pH. A sharp increase in the peak absorption maxima at 645 nm in the exhaled breath is indicative of a more acidic pH condition in the lungs and increased susceptibility to viral infection.

### Example 2

### Method for Administering Microparticles by Inhalation to a Human Subject for Monitoring a pH Condition in the Lung and Presence or Increased Susceptibility to an Inflammatory Condition in the Lungs of the Human Subject

A method and system is described for administering microparticles including a pH-responsive polymer or hydrogel, e.g., polymerized methacrylate, by inhalation to a human subject for monitoring a pH condition in the lung of the human subject. An acidic pH condition in the lungs can be associated with an inflammatory condition in the lung, e.g., asthma or chronic obstructive pulmonary disease (COPD). The acidic pH is at least partially due to activation of acid releasing inflammatory cells, e.g., neutrophils. The microparticles are configured to release a marker into the exhaled breath of a vertebrate subject when exposed to a pH condition in the lungs. The marker may be an olfactory marker, e.g., eugenol, which can be detected using an electronic odor sensor. See, e.g., U.S. Patent Application 2005/0233459, which is incorporated herein by reference.

The microparticles are formulated with a pH-responsive polymer or hydrogel, e.g., polymerized methacrylate, that will shrink or swell depending upon the pH conditions and allows for pH-controlled release of encapsulated materials. See, e.g., U.S. Pat. No. 7,642,328, which is incorporated herein by reference. To formulate the polymerized methacrylate, ethacrylate ester of oligomeric lactide is copolymerized with 4-vinyl pyridine to form a pH-sensitive hydrogel. The particle size, morphology, and texture are assessed as described above.

The pH-responsive microparticles are configured to encapsulate an olfactory marker, e.g., eugenol. Eugenol is a member of the phenylpropanoids and has an aroma of cloves. Methods for encapsulating eugenol in pH-responsive hydrogels, including methacrylate, are described. See, e.g., U.S. Pat. No. 7,144,957, which is incorporated herein by reference.

The pH-responsive microparticles including the olfactory marker, eugenol, are incorporated into an inhaler apparatus. The inhaler includes an electronic odor sensor array capable of sensing eugenol in exhaled breath following inhalation of the microparticles by the human subject. Electronic odor sensors capable of sensing eugenol have been described. See, e.g., U.S. Pat. No. 6,575,013; U.S. Patent Application 2005/0233459; Santos, et al., Sensors, 2004. Proceedings of IEEE, Oct. 24-27, 2004, pp. 341-344, each of which is incorporated herein by reference.

The inhaler includes an electronic odor sensor, which is a conductive polymer gas sensor consisting of a silicon substrate, gold plated electrodes, and a conducting organic polymer coating as the sensing element, operative at ambient temperature and capable of being included in the handheld inhaler apparatus (see, e.g., Bai & Shi, Sensors, 7:267-307, 2007; Wilson et al., IEEE Sensors Journal 1:256-274 each of which is incorporated herein by reference).

The human subject inhales the pH-sensitive microparticles containing the eugenol marker and the exhaled breath is analyzed. Increased eugenol in the expired breath of the subject as measured using the electronic odor sensor is indicative of decreased pH in the lung and exacerbation of a chronic inflammatory disease and may prompt changes to the subject's treatment regimen by the physician.

### Example 3

### Method for Administering Microparticles by Inhalation to a Human Subject for Monitoring Presence of a Bacterial Cell Surface Marker in the Lung and Presence or Increased Susceptibility to Bacterial Infection in the Lungs of the Human Subject

A method and system is described for administering microparticles by inhalation to a human subject for monitoring a bacterial infection in the human subject. Microparticles are configured to release a marker, e.g., water-soluble gold nanoparticles, into the exhaled breath of a human subject in response to an interaction with bacteria, e.g., Mycobacterium tuberculosis, in the lung of the human subject. The microparticles may be formulated from a hydrogel, at least two distinct but interacting oligonucleotide types holding the hydrogel together, and a marker loaded into the hydrogel. The hydrogel is formulated such that the integrity of the hydrogel and its contents is dependent upon the competing interaction of the oligonucleotides with one another relative to interaction with a target. See, e.g., Yang, et al., J. Am. Chem. Soc., 130:6320-6321, 2008, which is incorporated herein by reference. At least one of the oligonucleotide types include a specific DNA aptamer designed to recognize a bacterial cell surface marker. Upon binding to bacteria, e.g., M. tuberculosis, the DNA aptamer changes in configuration, disrupting the integrity of the hydrogel and allowing release of the marker, e.g., water-soluble gold nanoparticles, loaded into the hydrogel.

An aptamer for use in constructing the bacteria-responsive microparticles specific for M. tuberculosis may be generated using Systemic Evolution of Ligands by Exponential Enrichment (SELEX). See, e.g., Chen et al Biochem. Biophys. Res. Comm. 357: 743-748, 2007, which is incorporated herein by reference. One or more strains of M. tuberculosis (10⁸ colony forming units) are incubated with a combinatorial oligonucleotide library containing a set of single stranded DNA (ssDNA) oligonucleotides. The library may contain oligonucleotides of 20 to 30 nucleotides each, with all possible nucleotide combinations represented, providing a combinatorial library of approximately 10¹² to 10¹⁸ unique oligonucleotides. An iterative process is used to isolate oligonucleotides that selectively bind to the bacteria through multiple (e.g., 8-10) rounds of washing and screening.

The microparticles, formulated from a hydrogel material and target specific DNA aptamer, are generated essentially as described by Yang, et al, J. Am. Chem. Soc., 130:6320-6321, 2008, which is incorporated herein by reference. Two additional oligonucleotides (A and B) are designed to include nucleotide sequence that is complementary to and partially overlaps a portion of the M. tuberculosis DNA aptamer nucleotide sequence. The A and B oligonucleotides are further modified with phosphoramidite to form 5′ acryl-labeled oligonucleotides. The 5-acryl-labeled oligonucleotides are capable of co-polymerization with the hydrogel, acrylamide. The A and B oligonucleotides are separately co-polymerized with 4% acrylamide in the presence of ammonium persulfate and TEMED (tetramethylethylenediamine).

The bacteria-responsive microparticles may further include a marker released into the lung in response to interaction of the microparticles with M. tuberculosis. The marker is water-soluble gold nanoparticles (10-20 nm) capable of being carried in the exhaled water vapor upon release from the hydrogel. Gold nanoparticles are entrapped in the target-sensitive hydrogel during polymerization of the hydrogel. The gold particles are detected using absorbance spectroscopy at a constant wavelength of 620 nm. See, e.g., Yang, et al., J. Am. Chem. Soc., 130: 6320-6321, 2008, which is incorporated herein by reference.

The human subject inhales the bacteria-responsive microparticles having DNA aptamer and entrapped gold nanoparticles specific to M. tuberculosis. Upon interaction of the M. tuberculosis specific DNA aptamer with bacteria in the lung, the entrapped gold nanoparticles are released. The water-soluble gold nanoparticles are detected in the exhaled water vapor using absorbance spectroscopy. Upon detection of M. tuberculosis in the human subject the subject may be promptly isolated relative to other individuals and an appropriate antibiotic regimen may be initiated to treat the bacterial infection. In some aspects, the method described herein for detecting M. tuberculosis in the lungs of a human subject can be performed in a public place, e.g., prior to boarding a form of public transportation or prior to entering a crowded and enclosed space to prevent spread of infection to others.

### Example 4

### Method for Administering Microparticles by Inhalation to a Human Subject for Monitoring Presence of a Tumor Marker in the Lung and Presence or Increased Susceptibility to Lung Cancer of the Human Subject

A method and system is described for administering microparticles by inhalation to a human subject for detecting the presence of a neoplasia, e.g., lung cancer, in the human subject. The microparticles are configured to release a marker into the exhaled breath of a human subject in response to detecting a tumor antigen, e.g., CYFRA-21, in the lungs of the subject. CYFRA-21 is a fragment of cytokeratin 19 released from pulmonary epithelial cells and is elevated in the bronchoalveolar lavage (BAL) fluid of subjects with lung cancer. See, e.g., Prados, et al., Jpn. J. Clin. Oncol., 30:215-220, 2000, which is incorporated herein by reference. The microparticles may be formulated as a hydrogel with at least two distinct but interacting oligonucleotide types holding the hydrogel together, and an olfactory marker, vanillin, loaded into the hydrogel. The hydrogel is constructed such that the integrity of the hydrogel and its contents are dependent upon the competing interaction of the oligonucleotides with one another relative to interaction of the oligonucleotides with a target. See, e.g., Yang, et al., J. Am. Chem. Soc., 130:6320-6321, 2008, which is incorporated herein by reference. At least one of the oligonucleotide types includes a specific DNA aptamer designed to recognize CYRFA-21. Upon binding CYFRA-21, the DNA aptamer changes in configuration, disrupting the integrity of the hydrogel and allowing release of the olfactory marker vanillin from the hydrogel.

An aptamer configured to bind CYFRA-21 may be generated using Systemic Evolution of Ligands by Exponential Enrichment (SELEX). See, e.g., Wang et al., in J. Nanjing Medical University 21:277-281, 2007, which is incorporated herein by reference. Purified CYFRA-21 is immobilized on the surface of 96-well or 384-well plates and repeatedly screened and washed (8 to 10 cycles) against a biotinylated oligonucleotide library containing approximately 10¹² to 10¹⁸ unique oligonucleotides. Streptavidin-modified magnetic beads are used to concentrate CYFRA-21-specific aptamers.

The microparticles, are formulated as a hydrogel material and CYFRA-21-specific aptamer. See, e.g., Yang, et al. J. Am. Chem. Soc., 130:6320-6321, 2008, which is incorporated herein by reference. Two additional oligonucleotides (A and B) are designed to include nucleotide sequence that is complementary to and partially overlaps a portion of the CYFRA-21-specific aptamer nucleotide sequence. The A and B oligonucleotides are further modified with phosphoramidite to form 5′ acryl-labeled oligonucleotides. The 5-acryl-labeled oligonucleotides are capable of co-polymerization with the acrylamide hydrogel. The A and B oligonucleotides are separately co-polymerized with 4% acrylamide in the presence of ammonium persulfate and TEMED (tetramethylethylenediamine).

The CYFRA-21-responsive microparticles, formulated as a hydrogel material and CYFRA-21-specific aptamer, are further configured to contain an olfactory marker, e.g., vanillin. The olfactory marker vanillin is released from the microparticles upon interaction of the CYFRA-21-specific aptamer with CYFRA-21 in the lung of the human subject. An inhaler is used to administer the microparticles to the human subject. Analysis of vanillin released in the exhaled breath is performed using an electronic odor sensor array. The presence of vanillin detected by the electronic odor sensor array confirms the presence of a tumor in the lung of the subject. The electronic odor sensor is based on a tin oxide array composed of 16 thin film sensors doped with chromium and indium, operative at a temperature of 250° C. and capable of being included in a handheld sensor apparatus or as part of a larger diagnostic unit. See, e.g., Lozano, et al., Sensors, 2004. Proceedings of IEEE, Oct. 24-27, 2005, pp. 345-348; Wilson et al., IEEE Sensors Journal 1:256-274, 2001; U.S. Pat. No. 6,057,162, each of which is incorporated herein by reference). Additional diagnostic tools including imaging, bronchoscopy, and/or biopsy are used to confirm the presence of tumor in the lung of the subject.

### Example 5

### Method for Administering Microparticles by Inhalation to a Human Subject for Monitoring Presence of a Lung Tissue Condition and Presence or Increased Susceptibility to the Lung Tissue Condition of the Human Subject

A method and system is described for administering microparticles by inhalation to a human subject for time-controlled monitoring of a lung tissue condition, such as chronic inflammatory disease and/or increased susceptibility to a pulmonary infection, in a human subject. The time-controlled monitoring of the lung condition in the human subject can occur over minutes or hours. The microparticles are configured to include an inner layer and an outer layer. The inner layer of the microparticles is composed of a pH-responsive polymer or hydrogel, which upon exposure to a change in pH within the lung releases one or more olfactory markers, e.g., eugenol. The inner layer of the microparticles is encapsulated or coated by a polymer-based outer layer. The outer layer of the microparticles dissolves over time, exposing the pH-responsive inner layer in a time-dependent manner.

The pH-responsive inner layer of the microparticles is composed of a pH-responsive polymer or hydrogel, e.g., polymerized methacrylate, which shrinks or swells depending upon the pH conditions and allows for pH-controlled release of encapsulated materials. The pH-responsive methacrylate microparticles are synthesized. See, e.g., U.S. Pat. No. 7,642,328, which is incorporated herein by reference. Methacrylate ester of oligomeric lactide is copolymerized with 4-vinyl pyridine to form a pH-responsive hydrogel. An olfactory marker, e.g., eugenol, is incorporated into the pH-responsive hydrogel during the polymerization process. The particle size, morphology, and texture are assessed as described above.

The pH-responsive inner layer may be encapsulated or coated by an outer layer composed of a bio-degradable polymer that dissolves in a time-dependent manner. The time-controlled monitoring of the lung condition in the human subject can occur over minutes or hours. The pH-responsive inner layer is coated with poly(monostearoyl glycerol-co-succinate). See, e.g., U.S. Pat. No. 7,101,566, which is incorporated herein by reference. Poly(monostearoyl glycerol-co-succinate) is formed by combining monostearoyl glycerol, succinic anhydride, and stannous octoate and heating the solution under vacuum at a temperature of 180° C. The resulting co-polymer is dissolved in chloroform and placed into a fluidized coater (e.g., Precision Coater™, GEA Process Engineering, Inc., Columbia, Md.) along with the pH-responsive inner layer particles to generate poly(monostearoyl glycerol-co-succinate) coated pH-responsive microparticles. Increasing the thickness of the poly(monostearoyl glycerol-co-succinate) outer layer increases the length of time before exposure of the pH-responsive inner layer to the lung environment.

The microparticles formulated as a multi-layer of poly(monostearoyl glycerol-co-succinate) outer layer and a pH-responsive inner layer with the olfactory marker eugenol are incorporated into an inhaler apparatus. At time points following inhalation, an electronic odor sensor array associated with the inhaler apparatus or associated with a separate analyzing device is used to analyze the exhaled breath for the presence of the olfactory marker eugenol. The electronic odor sensor is a conductive polymer gas sensor consisting of a silicon substrate, gold plated electrodes, and a conducting organic polymer coating as the sensing element, operative at ambient temperature and capable of being included in a handheld inhaler apparatus. See, e.g., Bai & Shi, Sensors, 7:267-307, 2007; Wilson et al., IEEE

Sensors Journal 1:256-274 U.S. Pat. No. 6,575,013; U.S. Patent Application 2005/0233459; Santos, et al., Sensors, 2004. Proceedings of IEEE, Oct. 24-27, 2004, pp. 341-344; each of which is incorporated herein by reference. An increase in the olfactory marker eugenol in the expired breath of the subject, as measured using an electronic odor sensor, is indicative of decreased pH in the lung and exacerbation of a chronic inflammatory disease and/or increased susceptibility to a pulmonary infection in the human subject.

### Example 6

### Method for Administering Magnetic Microparticles by Inhalation to a Human Subject for Monitoring Presence of a Localized Lung Neoplasia and Presence or Increased Susceptibility to the Localized Lung Neoplasia in the Human Subject

A method and system is described for administering magnetic microparticles by inhalation to a human subject for detecting a localized lung tissue disease, e.g., a lung neoplasia, in the human subject. The microparticles are configured to include one or more magnetic particles to enable steering or accumulation of the magnetic microparticles in a particular location within the lung in response to an applied magnetic field. The magnetic microparticles are incorporated into target-responsive microparticles. The target-responsive microparticles are configured to release an olfactory marker, e.g., eugenol, into the exhaled breath of the subject in response to detecting and interacting with a tumor marker, e.g., carcinoembryonic antigen (CEA). CEA is a glycoprotein elevated in the bronchoalveolar lavage (BAL) fluid of subjects with lung cancer. See, e.g., Takahashi, et al., Jpn. J. Med. 24:236-243, 1985, which is incorporated herein by reference. The configuration described herein creates magnetically-steerable, target-responsive microparticles for monitoring tumors in localized regions of the lung.

The magnetically-steerable, CEA-responsive microparticles may be generated by incorporating magnetic microparticles and an olfactory marker, e.g., eugenol, into a CEA-responsive polymer. The magnetic microparticles are magnetite nanocrystals ranging in size from 2 nm to 20 nm and synthesized by mixing iron salts with alcohol, carboxylic acid and amine in an organic solvent and heating to 200-360° C. as described in U.S. Pat. No. 6,962,685, which is incorporated herein by reference. The magnetite nanocrystals as well as the eugenol, are incorporated into CEA-responsive microparticles during polymerization. An applied magnetic field is used to concentrate the magnetite-containing, CEA-responsive microparticles in a particular region of the lung.

The CEA-responsive microparticles are formulated using a hydrogel and processed by molecular imprinting. See, e.g., Miyata, et al., Proc. Natl. Acad. Sci., USA, 103:1190-1193, 2006, which is incorporated herein by reference. A lectin, e.g., concanavalin A (Con A), and anti-CEA antibody are separately conjugated with N-succinimidylacrylate to introduce polymerizable vinyl groups. The Con A acrylate and anti-CEA antibody acrylate are copolymerized with acrylamide in the presence of CEA. CEA is removed from the resultant network to generate a CEA-imprinted hydrogel. The hydrogel is loaded with the olfactory marker eugenol. Binding of CEA to the CEA-imprinted hydrogel causes swelling of the hydrogel and release of the olfactory marker.

An inhaler may be used to administer the magnetically-steerable, CEA-responsive microparticles by inhalation into the lungs of a human subject. An applied magnetic field is used to concentrate or direct the magnetic microparticles to a specific location in the king. A magnetic tip with a magnetic flux gradient of ∇B>100 T m⁻¹ is able to selectively direct inhaled magnetized microparticles down one of two airway channels. See, e.g., Dames, et al., Nature Nanotech. 2:495-499, 2007, which is incorporated herein by reference. In another example, an external magnet with a magnetic field of 0.3 T and a field gradient of 11 T/m is able to cause accumulation of magnetic microparticles to a specific body location in an animal subject. See, e.g., Fortin-Ripoche, et al., Radiology, 239:415-424, 2006, which is incorporated herein by reference. The accumulated magnetically-steerable, CEA-responsive microparticles release eugenol in response to detecting lung cancer antigen, CEA, in the localized area. Eugenol is analyzed in the exhaled breath using an electronic odor sensor array. The electronic odor sensor is based on a tin oxide array composed of 16 thin film sensors doped with chromium and indium, operative at a temperature up to 250° C. and capable of being included in a handheld sensor apparatus or as part of a larger diagnostic unit configured to detect the presence of the tumor in the localized region of the lung. See, e.g., Lozano, et al., Sensors, 2004. Proceedings of IEEE, Oct. 24-27, 2005, pp. 345-348; Wilson et al., IEEE Sensors Journal 1:256-274, 2001; U.S. Pat. No. 6,057,162, each of which is incorporated herein by reference. Additional diagnostic tools including imaging, bronchoscopy, and/or biopsy are used to confirm the presence of tumor in the localized region of the lung.

### Example 7

### Method for Administering Gas-Filled Microbubble Microparticle by Inhalation to a Human Subject for Monitoring Presence of a Localized Lung Tumor and Presence or Increased Susceptibility to the Localized Lung Tumor in the Human Subject

A method and system is described for administering microparticles by inhalation to a human subject for detecting a localized lung tissue disease, e.g., a lung neoplasia, in the human subject. The microparticles are gas-filled microbubbles configured to release their contents in response to focused ultrasound. The application of focused ultrasound enables disruption of the inhaled gas-filled microbubbles in a specific region of the lung. The gas-filled microbubbles are further configured to encapsulate nanoparticles. The nanoparticles are tumor marker-responsive polymers configured to release an olfactory marker, e.g., eugenol or vanillin, in response to detecting a tumor marker, e.g., carcinoembryonic antigen (CEA), in the lung of the subject. The combination of ultrasound-responsive microparticles with tumor-marker responsive nanoparticles enables diagnostic monitoring of a lung neoplasia in a very specific region of the lung of the subject.

The tumor marker-responsive nanoparticles may be configured to include a responsive binding site for a lung tumor marker, e.g., carcinoembryonic antigen (CEA). The CEA-responsive nanoparticles include a hydrogel loaded with an olfactory marker, e.g., eugenol or vanillin. The hydrogel is held together with at least two distinct but interacting oligonucleotide types. The hydrogel is constructed such that the integrity of the hydrogel and its contents is dependent upon the competing interaction of the oligonucleotides with one another relative to interaction of the oligonucleotides with a lung tumor target. See, e.g., Yang, et al., J. Am. Chem. Soc., 130:6320-6321, 2008, which is incorporated herein by reference. The interacting oligonucleotide types include at least one DNA aptamer designed to recognize the CEA marker on the lung tumor. Upon binding CEA, the aptamer changes in configuration, disrupting the integrity of the hydrogel and allowing release of the olfactory marker.

An aptamer configured to bind CEA is generated using Systemic Evolution of Ligands by Exponential Enrichment (SELEX). See, e.g., Wang et al., in J. Nanjing Medical University 21:277-281, 2007, which is incorporated herein by reference. Purified CEA is immobilized on the surface of 96-well or 384-well plates and repeatedly screened and washed (8 to 10 cycles) against a biotinylated oligonucleotide library containing approximately 10¹² to 10¹⁸ unique oligonucleotides. Streptavidin-modified magnetic beads are used to concentrate CEA-specific aptamers.

The microparticles, including a hydrogel material and CEA-specific aptamer, are generated. See, e.g., Yang, et al (J. Am. Chem. Soc., 130:6320-6321, 2008, which is incorporated herein by reference. Two additional oligonucleotides (A and B) are designed to include nucleotide sequence that is complementary to and partially overlaps a portion of the CEA-specific aptamer nucleotide sequence. The A and B oligonucleotides are further modified with phosphoramidite to form 5′ acryl-labeled oligonucleotides. The 5′ acryl-labeled oligonucleotides are capable of co-polymerization with the hydrogel, acrylamide. The A and B oligonucleotides are separately co-polymerized with 4% acrylamide in the presence of ammonium persulfate and TEMED (tetramethylethylenediamine). The hydrogel is loaded during this polymerization process with eugenol or vanillin. Binding of CEA to the CEA-imprinted hydrogel causes swelling of the hydrogel and release of the olfactory marker.

The CEA-responsive nanoparticles containing olfactory markers, eugenol or vanillin, are further incorporated into gas-filled microbubbles. Microbubbles containing nanoparticles are generated essentially as described by Chow et al, Mag. Res. Med. 63: 224-229, 2010, which is incorporated herein by reference. Microbubbles with incorporated CEA-responsive nanoparticles are generated using a double-emulsion procedure in which poly(D,L-lactide-co-glycolic acid) is combined with ethyl acetate (50:50) and the CEA-responsive nanoparticles, mixed by sonication, homogenized with poly(vinyl alcohol), and mixed with isopropyl alcohol. The microbubbles are formed and are collected by centrifugation, washed with hexane, and freeze-dried. Prior to use, the lyophilized microparticles are resuspended in water. The microbubbles containing the nanoparticles range in size from about 1 μm to about 25 μm, which is a particle size range sufficient to reach variable depths within the pulmonary tree.

The microparticles/microbubbles are administered to a human subject using an inhaler. Following inhalation, focused ultrasound is used to selectively energize and disrupt the microbubbles. Localized disruption of the microbubbles results in localized release of the CEA-responsive nanoparticles. Interaction of the CEA-responsive nanoparticles with CEA in a tumor localized to a portion of the lung results in release of the olfactory markers, eugenol or vanillin. The olfactory marker released in response to localized CEA is detected in the exhaled breath using an electronic nose sensor as described herein, indicating the presence of a CEA-containing tumor in the localized region of the lung of the subject. Additional diagnostic tools including imaging, bronchoscopy, and/or biopsy are used to confirm the presence of the tumor in the localized region of the lung of the subject.

Each recited range includes all combinations and sub-combinations of ranges, as well as specific numerals contained therein.

All publications and patent applications cited in this specification are herein incorporated by reference to the extent not inconsistent with the description herein and for all purposes as if each individual publication or patent application were specifically and individually indicated to be incorporated by reference for all purposes.

Those having ordinary skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having ordinary skill in the art will recognize that there are various vehicles by which processes and/or systems and/or other technologies disclosed herein can be effected (e.g., hardware, software, and/or firmware), and that the preferred vehicle will vary with the context in which the processes and/or systems and/or other technologies are deployed. For example, if a surgeon determines that speed and accuracy are paramount, the surgeon may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies disclosed herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those having ordinary skill in the art will recognize that optical aspects of implementations will typically employ optically-oriented hardware, software, and or firmware.

In a general sense the various aspects disclosed herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices disclosed herein, or a microprocessor configured by a computer program which at least partially carries out processes and/or devices disclosed herein), electrical circuitry forming a memory device (e.g., forms of random access memory), and/or electrical circuitry forming a communications device (e.g., a modem, communications switch, or optical-electrical equipment). The subject matter disclosed herein may be implemented in an analog or digital fashion or some combination thereof.

The herein described components (e.g., steps), devices, and objects and the description accompanying them are used as examples for the sake of conceptual clarity and that various configuration modifications using the disclosure provided herein are within the skill of those in the art. Consequently, as used herein, the specific examples set forth and the accompanying description are intended to be representative of their more general classes. In general, use of any specific example herein is also intended to be representative of its class, and the non-inclusion of such specific components (e.g., steps), devices, and objects herein should not be taken as indicating that limitation is desired.

With respect to the use of substantially any plural or singular terms herein, the reader can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an”; the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method comprising:
administering at least one microparticle to lungs of a vertebrate subject, wherein the at least one microparticle includes one or more markers; wherein the one or more markers is configured to be released in response to one or more physiological conditions in the vertebrate subject; and
detecting the one or more markers in a lung exhalant of the vertebrate subject.

2. The method of claim 1, further comprising determining the existence or absence of the one or more physiological conditions in the vertebrate subject.

3. The method of claim 1, wherein the one or more physiological conditions include at least one of lung tissue pH, lung chemical environment, lung tissue protein expression, lung bacterial occupation, or lung viral occupation.

4. The method of claim 1, wherein the one or more physiological conditions include one or more aspects of blood condition or blood chemistry.

5. The method of claim 1, wherein the one or more markers include at least one of a gas, vapor-phase chemical, gas-entrained aerosol, gas-entrained chemical, gas-entrained nanoparticle, or gas-entrained magnetic nanoparticle.

6.-8. (canceled)

9. The method of claim 1, wherein the one or more markers are embedded within the at least one microparticle or within an internal compartment of the at least one microparticle.

10. The method of claim 1, further comprising measuring the one or more markers in the lung exhalant based on at least one of odor, spectrometry, fluorescence spectrometry, chemical interaction, plasmon interaction, mass spectrometry, magnetism, or radioactivity.

11. The method of claim 1, wherein the at least one microparticle includes one or more of liposomes, microspheres, hydrogels, or porous nanoparticle-aggregate particles (PNAP).

12. The method of claim 1, wherein the at least one microparticle is configured to release the one or more markers in response to a lung tissue environment or lung cell interaction.

13.-14. (canceled)

15. The method of claim 1, further comprising providing one or more sensors configured to detect the one or more markers in the lung exhalant.

16. The method of claim 15, wherein the one or more sensors are in communication with one or more controllers.

17. The method of claim 16, wherein the one or more controllers are configured to regulate a time period for detecting the one or more markers in the lung exhalant from the vertebrate subject.

18. (canceled)

19. The method of claim 17, wherein the time period for detecting is a predetermined time after the administration of the at least one microparticle.

20. The method of claim 1, further comprising providing at least one chemical trigger or physical trigger configured to expose the one or more markers within the at least one microparticle.

21. The method of claim 20, wherein the at least one chemical trigger or physical trigger is configured to expose the one or more markers to the physiological conditions surrounding the at least one microparticle.

22.-24. (canceled)

25. The method of claim 20, further comprising providing one or more sensors that detect the one or more markers in the lung exhalant, wherein the one or more sensors is in communication with one or more controllers and wherein the one or more controllers are configured to activate the at least one chemical trigger or physical trigger to expose the one or more markers within the at least one microparticle for detection by the one or more sensors.

26. The method of claim 20, further comprising providing one or more sensors that detect the one or more markers in the lung exhalant, wherein the one or more sensors is in communication with one or more controllers and wherein the one or more controllers are configured to activate the at least one chemical trigger or physical trigger by transdermal energy deposition.

27. The method of claim 20, further comprising providing one or more sensors that detect the one or more markers in the lung exhalant, wherein the one or more sensors is in communication with one or more controllers and wherein the one or more controllers are configured to activate the at least one chemical trigger or physical trigger in a targeted lung region of the vertebrate subject.

28. The method of claim 1, further comprising providing the at least one microparticle having at least one hydraulic diameter configured to interact at one or more depths in the lung tissue of the vertebrate subject.

29. The method of claim 1, wherein the at least one microparticle includes at least one magnetic microparticle.

30. The method of claim 29, further comprising providing one or more magnetic elements configured to control motion of the at least one magnetic microparticle.

31. A device comprising:
an applicator configured to deliver at least one microparticle to lungs of a vertebrate subject, wherein the at least one microparticle includes one or more markers; and wherein the one or more markers is configured to be released in response to one or more physiological conditions in the vertebrate subject; and
one or more sensors configured to detect the one or more markers in a lung exhalant of the vertebrate subject.

32. The device of claim 31, wherein detection of the one or more markers in the lung exhalant is indicative of the existence or absence of the one or more physiological conditions in the vertebrate subject.

33. (canceled)

34. The device of claim 31, wherein the device including the applicator and the one or more sensors are in one unit.

35. The device of claim 31, wherein the device including the applicator and the one or more sensors are in two or more separate units.

36. The device of claim 31, wherein the one or more physiological conditions include at least one of lung tissue pH, lung chemical environment, cell surface receptor binding, endocytosis, cellular interaction with vertebrate cells, or cellular interaction with bacterial cells.

37. The device of claim 31, wherein the one or more physiological conditions include one or more aspects of blood condition or blood chemistry.

38. The device of claim 31, wherein the one or more markers include at least one of a gas, vapor-phase chemical, gas-entrained aerosol, gas-entrained chemical, or gas-entrained nanoparticle, or gas-entrained magnetic nanoparticle.

39. The device of claim 38, wherein the gas-entrained nanoparticle or the gas-entrained magnetic nanoparticle includes one or more quantum dots.

40. The device of claim 38, wherein the gas-entrained nanoparticle or the gas-entrained magnetic nanoparticle includes one or more of optically responsive components, dyes, fluorescent dyes, rare earth metals, or nonlinear optical materials.

41. The device of claim 38, wherein the gas-entrained nanoparticle or the gas-entrained magnetic nanoparticle includes one or more of plasmon responsive components, plasmon responsive metallic structure, or plasmon responsive metallic coating.

42. The device of claim 31, wherein the one or more markers are embedded within the at least one microparticle or within an internal compartment of the at least one microparticle.

43. The device of claim 31, wherein the one or more markers in the lung exhalant are configured to be measured based on at least one of odor, spectrometry, fluorescence spectrometry, chemical interaction, plasmon interaction, mass spectrometry, or radioactivity.

44. The device of claim 31, wherein the at least one microparticle includes one or more of liposomes, microspheres, hydrogels, or porous nanoparticle-aggregate particles (PNAP).

45. The device of claim 31, wherein the at least one microparticle is configured to release the one or more markers in response to a lung tissue environment or lung cell interaction.

46. The device of claim 45, wherein the at least one microparticle is configured to release the one or more markers based on at least one of lung tissue pH, lung chemical environment, cell surface receptor binding, endocytosis, interaction with vertebrate cells, interaction with bacterial cells, or interaction with viruses or viral-infected cells, in the lung tissue of the vertebrate subject.

47. The device of claim 45, wherein the at least one microparticle is configured to release the one or more markers based on one or more of blood condition or blood chemistry.

48. The device of claim 31, wherein the one or more sensors are in communication with one or more controllers.

49. The device of claim 48, wherein the one or more controllers are configured to regulate a time period for detecting the one or more markers in the lung exhalant from the vertebrate subject.

50. The device of claim 49, wherein the time period for detecting is substantially immediately upon sensing the exhalant or delayed upon sensing the exhalant.

51. The device of claim 49, wherein the time period for detecting is a predetermined time after the administration of the at least one microparticle.

52. The device of claim 31, further comprising at least one chemical trigger or physical trigger configured to expose the one or more markers within the at least one microparticle.

53. The device of claim 52, wherein the at least one chemical trigger or physical trigger is configured to expose the one or more markers to the physiological conditions surrounding the at least one microparticle.

54. The device of claim 52, wherein the at least one chemical trigger or physical trigger is a coating at least partially surrounding the at least one microparticle.

55. The device of claim 52, wherein the at least one chemical trigger or physical trigger is configured to be responsive to at least one of lung tissue pH, lung chemical environment, cell surface receptor binding, endocytosis, cellular interaction with vertebrate cells, or cellular interaction with bacterial cells, cellular interaction with viruses or viral-infected cells, or transdermal energy deposition, in the lung tissue of the vertebrate subject.

56. The device of claim 52, further comprising one or more controllers configured to activate the at least one chemical trigger or physical trigger to expose the one or more markers within the at least one microparticle for detection by the one or more sensors.

57. The device of claim 56, wherein the one or more controllers are configured to activate the at least one chemical trigger or physical trigger by transdermal energy deposition.

58. The device of claim 56, wherein the one or more controllers are configured to activate the at least one chemical trigger or physical trigger in a targeted lung region of the vertebrate subject.

59. The device of claim 31, wherein the at least one microparticle having at least one hydraulic diameter is configured to interact at one or more depths in the lung tissue of the vertebrate subject.

60. The device of claim 59, wherein the at least one microparticle includes at least one magnetic microparticle.

61. The device of claim 60, further comprising one or more magnetic elements configured to control motion of the at least one magnetic microparticle.

62. The device of claim 61, wherein the magnetic element is a separate unit from the applicator and the one or more sensors.

63. The device of claim 31, further comprising one or more controllers configured to regulate a time period for detection of the one or more markers in the lung exhalant of the vertebrate subject.

64. The device of claim 63, wherein the time period for detection is immediately upon sensing the exhalant or delayed upon sensing the exhalant.

65.-107. (canceled)
